(19) United States
(12) Patent Application Publication
(10) Pub. No.: US 2014/0302152 A1
(43) Pub. Date: Oct. 9, 2014

(54) GRANULATES COMPRISING ESLICARBAZEPINE ACETATE

(71) Applicant: Jose Pedro Pelxoto Rito Pontes, Braga (PT)

(72) Inventors: Pedro Miguel Da Costa Barrocas, Valongo (PT); Ricardo Jorge Dos Santos Lima, Ermesinde (PT); Teofilo Cardoso De Vasconcelos, Maia (PT); Ligia Sofia De Castro Pereira, S. Mamede do Coronado (PT); Paula Cristina De Almeida Jeronimo, Basel (CH); Rui Cerdeira De Campos Costa, Basel (CH)

(73) Assignee: BIAL-PORTELA & CA., S.A., S. Mamede do Coronado (PT)

(21) Appl. No.: 13/977,894

(22) PCT Filed: Dec. 30, 2011
(86) PCT No.: PCT/PT2011/000048

§ 371 Date: Jun. 12, 2014

Related U.S. Application Data
(60) Provisional application No. 61/428,905, filed on Dec. 31, 2010.

(51) Int. Cl. A61K 9/16; A61K 31/55

(52) U.S. Cl. CPC A61K 9/1682; A61K 31/55
USPC 424/490; 424/489; 514/217; 427/2.14

(57) ABSTRACT

The invention relates to a solid pharmaceutical composition, the composition comprising eslicarbazepine acetate and one or more pharmaceutically acceptable excipients, wherein the composition is in the form of granules, and wherein at least 90% of the granules of the composition have a particle size of 90 μm or more, and/or wherein at least 50% of the granules of the composition have a particle size of 250 μm or more. The invention also relates to a process for producing a granular composition. Further, the invention relates to the use of the composition in therapy and, in particular, in the treatment or prevention a disorder selected from epilepsy, neuropathic pain, migraine, fibromyalgia an affective disorders.

### FIELD OF THE INVENTION

The present invention relates to a solid pharmaceutical composition comprising eslicarbazepine acetate (ESL), wherein the composition is in the form of granules, and wherein at least 90% of the granules of the composition have a particle size of about 90 μm or more, and/or wherein at least 50% of the granules of the composition have a particle size of about 250 μm or more. The present invention also relates to a process for producing a granular composition comprising a pharmaceutically active agent, wherein at least 90% of the granules that are produced have a particle size of about 90 μm or more and/or wherein at least 50% of the coated granules that are produced have a particle size of about 250 μm or more.

### BACKGROUND OF THE INVENTION

WO2009/054743 relates to oral compositions of eslicarbazepine acetate and methods of making them. However, this document does not disclose granular compositions in which at least 90% of the granules of the composition have a particle size of at least about 90 μm, and/or wherein at least 50% of the granules of the composition have a particle size of at least about 250 μm.

### SUMMARY OF THE INVENTION

In a first aspect, the present invention provides a solid pharmaceutical composition for oral administration, the composition comprising eslicarbazepine acetate and one or more pharmaceutically acceptable excipients, wherein the composition is in the form of granules, and wherein at least 90% of the granules of the composition have a particle size of at least about 90 μm, and/or wherein at least 50% of the granules of the composition have a particle size of at least about 250 μm.

The granules of the composition are larger than known particles of eslicarbazepine acetate. An advantage provided by this is that the granules dissolve more slowly. This means that it is less likely that a subject taking the granules will experience an unpleasant taste associated with one of the ingredients in the composition. For example, the composition can be applied to food which is then eaten by the subject. The larger granules will dissolve more slowly on the food so that, when the food is eaten, the food is less likely to have an unpleasant taste as a result of one of the components of the composition, for example, the eslicarbazepine acetate.

The granules of the composition are relatively homogeneous in size, i.e. the range of particle sizes in the composition is relatively narrow. This means that the granules are easier to use because, for example, they can be sprinkled onto food more easily and more evenly.

Granules are also easier to use in manufacturing processes. For example, they can be weighed more easily. They are also easier to fill into sachets. Further, granules are easier to pour, for example, out of a sachet. This means that they can be administered more easily and reduces wastage compared to a powder which may adhere to surfaces undesirably.

The term “granule” means a particle which is a permanent aggregate (i.e. remaining substantially or completely in aggregated form following granulation using granulation liquids and drying) formed from a number of smaller particles. Generally, the smaller particles can still be identified in the particle forming the granule. The term granule does not imply any limitation on the size of the particle forming the granule. However, as discussed in more detail below, the granules of the invention may have particular limitations on the particle size of the granules. In accordance with the invention, the granules of the composition, or at least those granules having a particle size of 90 μm or more, each contain eslicarbazepine acetate and one or more pharmaceutically acceptable excipients. These are formed by a granulation process in which the eslicarbazepine acetate and one or more pharmaceutically acceptable excipients are granulated together to increase the overall particle size of the components. Preferably, the granules (i.e. each granule) will comprise a binder which helps to aggregate and maintain the eslicarbazepine acetate and one or more pharmaceutically acceptable excipients in the form of a granule so that it is less likely to break up into smaller particles.

In one embodiment, at least 90% of the granules of the composition have a particle size of at least about 90 μm. In other embodiments, at least 90% of the granules of the composition have a particle size of at least about 100, 110, 120, 130, 140, 150, 160, 170, 180, 190, 200, 210, 220, 230, 240 or 250 μm.

In another embodiment, at least 50% of the granules of the composition have a particle size of at least about 250 μm. In other embodiments, at least 50% of the granules of the composition have a particle size of at least about 260, 270, 280, 290, 300, 310, 320, 330, 340, 350, 360, 370, 380, 390, 400 or 420 μm.

In a further embodiment, at least 90% of the granules of the composition may have a particle size of about 1600 μm or less. Alternatively, at least 90% of the granules of the composition may have a particle size of about 1550, 1500, 1450, 1400, 1350, 1300, 1250, 1200, 1150, 1100, 1050, 1000, 950, 900, 850, 800, 750, 700, 650, 600, 550, 500, 450, 420, 400, 350 or 300 μm or less.

In an alternative embodiment, at least 80% of the granules of the composition have a particle size which falls within a range of about 2000 μm. In other embodiments, at least 80% of the granules of the composition have a particle size which falls within a range of about 1800, 1700, 1600, 1500, 1450, 1400, 1350, 1300, 1250, 1200, 1150, 1100, 1050, 1000, 950, 900, 850, 800, 750, 700, 650, 600, 550, 500, 450, 400, 350, 300, 250, 200, 150 or 100 μm.

The measurement of the particle size of the granules and the distribution of particle sizes can easily be measured by one skilled in the art who would know appropriate methods for determining these parameters. For example, a sieve battery or laser diffraction can be used for obtaining such measurements.

The composition comprises eslicarbazepine acetate (IUPAC name: (S)-10-Acetoxy-10,11-dihydro-5H-dibenz[b,f]azepine-5-carboxamide) which is well known to those skilled in the art and methods for synthesising eslicarbazepine acetate are also well known, for example from U.S. Pat. No. 5,753,646.

In one embodiment, the composition comprises between about 2% and about 98% by weight of eslicarbazepine acetate. In certain such embodiments, the composition comprises at least about 3%, 4%, 5%, 6%, 7%, 8%, 9%, 10%, 11%, 12%, 13%, 14% or 15% eslicarbazepine acetate by weight. In certain such embodiments, the composition comprises up to about 85%, 70%, 60%, 50%, 40%, 35%, 30%, 25%, 20% or 15% eslicarbazepine acetate by weight. In particular embodiments, the amount of eslicarbazepine acetate in the composition by weight may be between about 5% and about 85%, between about 7% and about 70%, between about 10% and about 50%, between about 5% and about 25%, or between about 5% and about 15%.

The composition may comprise filler material. In one embodiment, the composition comprises between about 2% and about 98% filler material by weight. In certain such embodiments, the composition comprises at least about 5%, 10%, 15%, 20%, 25%, 30%, 35%, 40%, 45%, 50%, 55%, 60%, 65%, 70% or 75% filler material by weight. In certain such embodiments, the compositions comprises up to about 95%, 90%, 85%, 80% or 75% filler material by weight. In certain embodiments, the composition comprises between about 15% and about 95%, between about 30% and about 90%, between about 50% and about 80%, between about 60% and about 90%, or between about 70% and about 80% filler material by weight.

The filler material may be any pharmaceutically acceptable filler material. A skilled person is well aware of conventional filler material which is employed in the field of pharmaceutical formulation. For example, the filler material may be selected from microcrystalline cellulose, anhydrous lactose, Cellactose® 80 (co-processed 75% microcrystalline cellulose and 25% lactose), isomalt, dibasic dihydrate calcium phosphate, calcium carbonate, calcium lactate, dibasic anhydrous calcium phosphate, tribasic calcium phosphate, calcium silicate, calcium sulfate, carbomer, carboxymethylcellulose calcium, carboxymethylcellulose sodium, cellulose, silicified microcrystalline cellulose, cellulose acetate, ceratonia, chitosan, copovidone, corn starch, pregelatinized starch, dextrates, dextrin, dextrose, erythritol, ethylcellulose, fructose, fumaric acid, glyceryl monooleate, glyceryl monostearate, glyceryl palmitostearate, hydroxyethyl cellulose, hydroxyethylmethyl cellulose, hydroxypropyl betadex, hydroxypropyl cellulose, hydroxypropyl starch, hypromellose, hypromellose acetate succinate, kaolin, lactitol, anhydrous lactose, lactose monohydrate, magnesium carbonate, magnesium oxide, maltitol, maltodextrin, maltose, mannitol, methylcellulose, pectin, polaxamer, polycarbophil, polydextrose, poly(DL-lactic acid), polyethylene glycol, polyethylene oxide, polymethacrylates, polyoxyglycerides, polyvinyl alcohol, povidone, shellac, simethicone, sodium alginate, sodium chloride, sorbitol, starch, pregelatinized starch, sucrose, sugar spheres, sulfobutylether B-cyclodextrin, titanium dioxide, trehalose, microcrystalline wax, white wax, yellow wax, xanthan gum, xylitol, and zein, or any combination thereof.

In certain embodiments, the filler material is selected from one or more of lactose, dibasic dihydrate calcium phosphate and isomalt. Preferably, the filler material is: lactose and dibasic dihydrate calcium phosphate; or isomalt and dibasic dihydrate calcium phosphate; or lactose and isomalt. More preferably, the filler material is lactose and dibasic dihydrate calcium phosphate.

When the filler material comprises lactose, the composition preferably comprises between about 5% and about 90% lactose by weight. In other embodiments, the composition comprises at least about 10%, 15%, 20%, 25%, 30%, 35%, 40%, 45% or 50% lactose by weight. In certain embodiments, the composition comprises up to about 85%, 80%, 75%, 70%, 65%, 60% or 55% lactose by weight. In particular embodiments, the amount of lactose in the composition by weight may be between about 5% and about 80%, between about 15% and about 75%, between about 25% and about 60% or between about 40% and about 60%.

When the filler material comprises dibasic dihydrate calcium phosphate, the composition preferably comprises between about 10% and about 50% dibasic dihydrate calcium phosphate by weight. More preferably, the amount of dibasic dihydrate calcium phosphate in the composition by weight is between about 15% and about 50%, between about 10% and about 35%, between about 15% and about 30%, between about 15% and about 25% or between about 20% and about 25%.

When the filler material comprises isomalt, the composition preferably comprises between about 5% and about 90% isomalt by weight. In certain such embodiments, the composition comprises at least about 10%, 15%, 20%, 25%, 30%, 35%, 40%, 45% or 50% isomalt by weight. In certain embodiments, the composition comprises up to about 85%, 80%, 75%, 70%, 65%, 60% or 55% isomalt by weight. In particular embodiments, the amount of isomalt in the composition by weight may be between about 5% and about 80%, between about 15% and about 75%, between about 25% and about 60% or between about 40% and about 60%.

The composition may comprise a binder. The binder may be any pharmaceutically acceptable binder. A skilled person is well aware of conventional binders which are employed in the field of pharmaceutical formulation. For example, the binder may be selected from acacia, agar, povidone, alginic acid, calcium alginate, calcium carbonate, calcium lactate, carbomer, carboxymethylcellulose calcium, carboxymethylcellulose sodium, carrageenan, microcrystalline cellulose, cellulose acetate phthalate, ceratonia, ceresin, chitosan, copovidone, corn starch, pregelatinized starch, crospovidone, cottonseed oil dextrates, dextrin, dextrose, ethylcellulose, gelatin, glyceryl behenate, guar gum, hydroxyethyl cellulose, hydroxyethylmethyl cellulose, hydroxypropyl cellulose, hydrogenated vegetable oil type I, hydroxypropyl starch, hypromellose, hypromellose acetate succinate, hypromellose phthalate, inulin, isomalt, lactose, liquid glucose, magnesium aluminium silicate, maltodextrin, maltose, mannitol, methylcellulose, pectin, polaxamer, polycarbophil, polydextrose, polyethylene oxide, polymethacrylates, sodium alginate, starch, stearic acid, sucrose, sunflower oil, tricaprylin, vitamin E polyethylene glycol succinate, xanthan gum, and zein, or any combination thereof.

In one embodiment, the binder is selected from xanthan gum, HPMC, starch, sodium alginate and povidone. Preferably, the binder is povidone.

The composition may comprise any suitable amount of binder. In a particular embodiment, the composition comprises between about 2% and about 15% binder by weight. In other embodiments, the amount of binder in the composition by weight may be between about 5% and about 12% or between about 6% and about 10%.

In certain embodiments, the composition may further comprise a colouring agent. Further, the colouring agent may be distributed in the composition so that the granules have a homogeneous colour across their cross section. This allows the process used to produce the granules to be assessed as to whether it has been carried out correctly. For example, if the colouring agent is not distributed in the composition so that the granules have a homogeneous colour across their cross section, this is an indication that the production process has not been carried out correctly. This indicator is relatively easy to detect compared to carrying out tests on the granules. Therefore, any problems in the production process can be identified relatively easily and quickly.

The granules may also have a homogeneous colour as a whole so that each granule is substantially the same colour as the other granules. Again, this allows quick and easy assessment of the production process. If not all the granules have a homogeneous colour, this can indicate a problem with the production process.

A further advantage of all the granules having a homogeneous colour is that it makes the granules more appealing to a subject, particularly as it masks damaged, i.e. broken, granules. This means that the granules are likely to be more acceptable to the subject and may help with patient compliance.

Accordingly, in certain embodiments, the invention relates to a method for assessing the quality of the process used to prepare the granules as disclosed herein, comprising evaluating the homogeneity of colour distribution within (cross-sectionally) and/or between the granules of the composition.

The homogeneity of the colouration of the granules (both the cross-section and homogeneity between granules) can be measured using any suitable method known to those skilled in the art. For example, the colour homogeneity can be measured using colorimetry.

For colorimetric determinations, a colorimeter can be used, for example, a Jasco V-650 CFR with colour diagnosis software. This equipment can present the results in different colour systems:
CIE 1931 XYZ colour space in which each character XYZ represents respectively red, green and blue—proposed by CIE (International Commission on Illumination) in 1931; or
CIE 1976 L*a*b* colour space in which L* represents the lightness of the colour, a* a position between red/magenta and green and b* a position between yellow and blue—proposed by CIE in 1976. This is the preferred method of displaying the results.

With this equipment, the L*a*b* values can be obtained of several samples of the same batch. This would allow determination of the homogeneity of the colouration of the samples.

There is also a USP test (<1061> COLOR-INSTRUMENTAL MEASUREMENT. See United States Pharmacopoeia 31, The National Formulary 26, 2008, Rockville) which details the type of instrument and the conversion between the above two colour systems. This is detailed below:

The observed colour of an object depends on the spectral energy of the illumination, the absorbing characteristics of the object, and the visual sensitivity of the observer over the visible range. Similarly, it is essential that any instrumental method that is widely applicable take these same factors into account.

The basis of any instrumental measurement of colour is that the human eye has been shown to detect colour via three “receptors.” Hence, all colours can be broken down into a mixture of three radiant stimuli that are suitably chosen to excite all three receptors in the eye. Although no single set of real light sources can be used to match all colours (i.e., for any three lights chosen, some colours require a negative amount of one or more of the lights), three arbitrary stimuli have been defined, with which it is possible to define all real colours. Through extensive colour-matching experiments with human subjects having normal colour vision, distributing coefficients have been measured for each visible wavelength (400 nm to 700 nm) giving the relative amount of stimulation of each receptor caused by light of that wavelength. These distribution coefficients x, y, z, are shown below. Similarly, for any colour the amount of stimulation of each receptor in the eye is defined by the set of Tristimulus values (X, Y, and Z) for that colour.

The relationships between the distribution coefficient (see FIG. 1) and the tristimulus values are given in the equations:

X=∫₀^{∞}f_{λ} x̅_{λ}P_{λ}dλ/Y′,

Y=∫₀^{∞}f_{λ} y̅_{λ}P_{λ}dλ/Y′, and

Z=∫₀^{∞}f_{λ} z̅_{λ}P_{λ}dλ/Y′,

in which

Y′=∫₀^{∞}f_{λ} y̅_{λ}P_{λ}cλ,P_{λ},

is the spectral power of the illuminant, and f_{λ} is either the spectral reflectance (Σ_{λ}) or spectral transmittance (τ_{λ}) of the material.

Once the tristimulus values of a colour have been determined, they may be used to calculate the coordinates of the colour in an idealized three-dimensional colour space referred to as a visually uniform colour space. Many sets of colour equations have been developed in an attempt to define such a space. The equations given herein represent a compromise between simplicity of calculation and conformance with ideality.

The coordinates of a colour in a visually uniform colour space may be used to calculate the deviation of a colour from a chosen reference point. Where the instrumental method is used to determine the result of a test requiring colour comparison of a test preparation with that of a standard or matching fluid, the parameter to be compared is the difference, in visually uniform colour space, between the colour of the blank and the colour of the test specimen or standard.

### Procedure

In a spectrophotometric method, reflectance or transmittance values are obtained at discrete wavelengths throughout the visible spectrum, a band width of 10 nm or less being used. These values are then used to calculate the tristimulus values through the use of weighting factors (Typical weighting factors are given by ASTM Z58.7.1-1951 as reported in the Journal of the Optical Society of America, Vol. 41, 1951, pages 431-439). In a colorimetric method, the weighting is performed through the use of filters.

In the measurement of the spectral reflectance of opaque solids, the angle of viewing is separated from the angle of illumination in such a manner that only rays reflected diffusely from the test specimen enter the receptor. Specular reflection and stray light are excluded.

For the measurement of the spectral transmittance of clear liquids, the specimen is irradiated from within 5 degrees of the normal to its surface, and the transmitted energy measured is that confined within 5 degrees from the normal. The colour of solutions changes with the thickness of the layer measured. Unless special considerations dictate otherwise, a layer 1 cm thick should be used. The methods described here are not applicable to hazy liquids or translucent solids.

### Calibration

For purposes of calibration, one of the following reference materials may be used, as required by instrument geometry. For transmittance measurements, purified water may be used as a white standard and assigned a transmittance of 1.000 at all wavelengths. Then the tristimulus values X, Y, and Z for CIE source C are 98.0, 100.0, and 118.1, respectively. For reflectance measurements, opaque porcelain plaques, whose calibration base is the perfect diffuse reflector and whose reflectance characteristics have been determined for the appropriate instrumental geometry, may be used (Suitable items are available from BYK-Gardner USA, 2431 Linden Lane, Silver Spring, Md. 20910, or from Hunter Associates Laboratory, Inc., 11491 Sunset Hills Road, Reston, Va. 22090). If the geometry of sample presentation precludes the use of such plaques, pressed barium sulfate, white reflectance standard grade, may be used (Suitable material is available from Eastman Kodak Company, Rochester, N.Y. 14650, as “White Reflectance Standard.”). After calibration with the above-mentioned materials, it is desirable whenever possible to measure a reference material as close to the colour of the sample as possible. If a sample of the material being tested is not suitable for use as a long-term standard, colour chips are available (Centroid Colour Charts may be obtained from suppliers of instruments for measurement of colour) which span the entire visually uniform colour space in small increments. The use of such a reference standard is encouraged as a means of monitoring instrument performance even for absolute colour determinations.

### Spectrophotometric Method

The reflectance or transmittance from 380 to 770 nm may be determined at intervals of 10 nm and the results expressed as a percentage, the maximum being 100.0. The tristimulus values X, Y, and Z may then be calculated as follows.

### Reflecting Materials

For reflecting materials the quantities X, Y, and Z are

X=Σ₃₈₀⁷⁷⁰ρ_{λ} x̅_{λ}P_{λ}Δλ/Y′,

Y=Σ₃₈₀⁷⁷⁰ρ_{λ} y̅_{λ}P_{λ}Δλ/Y′, and

Z=Σ₃₈₀⁷⁷⁰ρ_{λ} z̅_{λ}P_{λ}Δλ/Y′

in which

Y′=Σ₃₈₀⁷⁷⁰ y̅_{λ}P_{λ}Δλ,ρ_{λ}

is the spectral reflectance of the material,
x̅_{λ}P_{λ}, y̅_{λ}P_{λ}, and z̅_{λ}P_{λ}
are known values associated with each Standard Source (Typical weighting factors are given by ASTM Z58.7.1-1951 as reported in the Journal of the Optical Society of America, Vol. 41, 1951, pages 431-439, and suitable items are available from BYK-Gardner USA, 2431 Linden Lane, Silver Spring, Md. 20910, or from Hunter Associates Laboratory, Inc., 11491 Sunset Hills Road, Reston, Va. 22090) and Δλ is expressed in nm.

### Transmitting Materials

For transmitting materials, the quantities X, Y, and Z are calculated as above, τ_{λ} (spectral transmittance) being substituted for ρ_{λ}.

### Colorimetric Method

A suitable colorimeter (A suitable tristimulus colorimeter is available from BYK-Gardner USA, 2431 Linden Lane, Silver Spring, Md. 20910, or from Hunter Associates Laboratory, Inc., 11491 Sunset Hills Road, Reston, Va. 22090) may be operated to obtain values equivalent to the tristimulus values, X, Y, and Z. The accuracy with which the results obtained from the filter colorimeter match the tristimulus values may be indicated by determining the tristimulus values of plaques of strongly saturated colours and comparing these values with those computed from spectral measurements on a spectrophotometer.

### Interpretation

### Color Coordinates

The Colour Coordinates, L*, a*, and b* are defined by

L*=116(Y/Yₒ)^{1/3}−16,

a*=500[(X/Xₒ)^{1/3}−(Y/Yₒ)^{1/3}], and

b*=200[(Y/Yₒ)^{1/3}−(Z/Zₒ)^{1/3}]

in which Xₒ, Yₒ, and Zₒ are the tristimulus values of the nominally white or colourless standard, and Y/Yₒ>0.01. Usually they are equal to the tristimulus values of the standard illuminant, with Yₒ set equal to 100.0. In this case Xₒ=98.0 and Zₒ=118.1.

### Color Difference

The total Colour Difference ΔE* is

ΔE*=[(ΔL*)²+(Δa*)²+(Δb*)²]^{1/2}

in which ΔL*, Δa*, and Δb* are the differences in colour coordinates of the specimens being compared. Instrumental variables can influence results.

The colour of a granule or granules is considered to be homogeneous if the colour difference (ΔE* (as defined above)) between the two colours which are furthest apart in colour space, occurring at particular points on or in the granule or granules, is less than 2.0. This means that there may be a small degree of variation in the colour of a granule or granules although this may not be particularly noticeable or may not be noticeable at all to the human eye. The colour difference (ΔE*) may be tested in the manner described above, for example, using USP test 1061. Preferably, the colour difference (ΔE*) is less than about 1.9, 1.8, 1.7, 1.6, 1.5, 1.4, 1.3, 1.2, 1.1, 1.0, 0.9, 0.8, 0.7, 0.6 or 0.5.

The colouring agent may be any pharmaceutically acceptable colouring agent which provides colour to the granules. A skilled person is well aware of conventional colouring agents which are employed in the field of pharmaceutical formulation. For example, the colouring agent may comprise a pigment selected from calcium carbonate, iron oxides, lakes, titanium oxide, caramel, allura red ac, amaranth, anthocyanins, azorubine, beetroot red, canthanxanthin, carmine, D&C red 33, Eosine YS, erythrosine, lithol rubine, phloxine B, ponceau 4R, Red 2G, beta-carotene, carotenes, curcumin, D&C yellow 10, quinoline yellow WS, riboflavin, Sunset yellow FCF, tartrazine, chlorophylls and chlorophyllins, Cu complexes of chlorophylls and chlorophyllins, fast green FCF, green S, brilliant blue FCF, indigotine, patent blue V, brilliant black BN, and vegetable carbon, or a combination thereof. In one embodiment, the colouring agent is a red colouring agent such as Opadry 31K250002 red or AquaPolish D RED. The colouring agent may also comprise a plasticiser, an adhesive, and optionally, a base. Lubricant(s) may also be added to the colouring agent. For example, a plasticizer and/or an adhesive may help the colouring agent stick to the excipients and/or outside of the granules to form a homogeneously coloured granule and/or coating. Suitable plasticisers, adhesives, bases and lubricants are well known to the skilled person but may be selected from the lists below.

Plasticisers may be selected from acetyltributyl citrate, benzyl benzoate, chlorbutanol, dextrin, dibutyl phthalate, dibutyl sebacate, diethyl phthalate, dimethyl phthalate, glycerin, glycerin monostearate, mannitol, mineral oil, lanolin alcohols, palmitic acid, petrolatum, polyethylene glycol, polyvinyl acetate, polyvinyl acetate phthalate, propylene glycol, pyrrolidone, sorbitol, stearic acid, triacetin, tributyl citrate, triethanolamine, and triethyl citrate, or a mixture of two or more thereof.

Adhesives may be selected from carbomers, dextrin, hypromellose, and poly(methylvinylether/maleic anhydride), or a mixture of two or more thereof.

Bases may be selected from acetyltriethyl citrate, calcium carbonate, carboxymethylcellulose calcium, carboxymethylcellulose sodium, carnauba wax, cellulose acetate, cellulose acetate phthalate, ceresin, cetyl alcohol, chitosan, ethylcellulose, fructose, gelatin, glycerin, glyceryl behenate, glyceryl palmitostearate, hydroxyethyl cellulose, hydroxyethylmethyl cellulose, hydroxypropyl cellulose, hypromellose, hypromellose phthalate, isomalt, latex particles, glucose, lactose, maltitol, maltodextrin, methylcellulose, microcrystalline wax, paraffin, poloxamer, polydextrose, polyethylene glycol, polyethylene oxide, poly-DL-(lactic acid), polyvinyl acetate phthalate, polyvinyl alcohol, potassium chloride, povidone, shellac, starch and its derivates, sucrose, titanium oxide, tributyl citrate, triethyl citrate, vanillin, white wax, xylitol, and yellow wax, or a mixture of two or more thereof.

Lubricants may be selected from calcium stearate, colloidal silicon dioxide, glyceryl behenate, glyceryl monostearate, glyceryl palmitostearate, leucine, magnesium oxide, magnesium silicate, magnesium stearate, magnesium trisilicate, myristic acid, palmitic acid, polaxamer, polyethylene glycol, sodium benzoate, sodium lauryl sulfate, sodium stearyl fumarate, stearic acid, talc, hydrogenated vegetable oil, and zinc stearate, or a mixture of two or more thereof.

The composition may comprise between about 1% and about 20% colouring agent by weight. In certain such embodiments, the amount of colouring agent in the composition by weight may be between about 1% and about 15%, between about 1% and about 10%, or between about 4% and about 8%.

The composition may further comprise a flavouring agent. The flavouring agent may be any pharmaceutically acceptable flavouring agent. A skilled person is well aware of conventionally flavouring agents which are employed in the field of pharmaceutical formulation. For example, the flavouring agent may be selected from chocolate, bubble gum, cocoa, coffee, fruit flavouring (such as wild cherry, strawberry, banana, grape, peach, and raspberry), oil of peppermint, oil of spearmint, oil of orange, mint flavour, anise flavour, honey flavour, vanilla flavour, tea flavour, verbena flavour, and various fruit acids such as citric acid, ascorbic acid and tartaric acid, and mixtures thereof. The composition may comprise between about 0.05% and about 5% flavouring agent by weight.

The composition may further comprise a sweetener. The sweetener may be any pharmaceutically acceptable sweetener. A skilled person is well aware of conventional sweeteners which are employed in the field of pharmaceutical formulation. For example, the sweetener may be selected from acesulfame potassium, aspartame, sucrose, sucralose, saccharin sodium, sugar, dextrose, fructose, mannitol, xylitol, alitame, glucose, lactilol, maltitol, maltose, sodium cyclamate, sorbitol, gluconate, and cyclamate and mixtures thereof. The composition may comprise between about 0.1% and about 10% sweetener by weight.

In a particular embodiment, the invention provides a solid pharmaceutical composition for oral administration, the composition comprising eslicarbazepine acetate and one or more pharmaceutically acceptable excipients, wherein the composition is in the form of granules, and wherein at least 90% of the granules of the composition have a particle size of 90 μm or more, and/or wherein at least 50% of the granules of the composition have a particle size of 250 μm or more, wherein the composition comprises between about 5% and 15% by weight of eslicarbazepine acetate, between about 70% and about 80% filler material by weight, between about 2% and about 15% povidone by weight, and between about 1% and about 10% colouring agent by weight, wherein the filler material comprises lactose and dibasic dihydrate calcium phosphate, wherein the composition comprises between about 40% and about 60% lactose by weight and between about 15% and about 30% dibasic dihydrate calcium phosphate by weight, and wherein the granules have a homogeneous colour across their cross section.

As will be appreciated by a person skilled in the art, the one or more pharmaceutically acceptable excipients may be any suitable excipients which can be used in the field of pharmaceutical formulation. For example, the one or more pharmaceutically acceptable excipients may be a pharmaceutically acceptable carrier, adjuvant or vehicle. Pharmaceutically acceptable carriers, adjuvants and vehicles that may be used in the pharmaceutical compositions of this invention are those conventionally employed in the field of pharmaceutical formulation, and include, but are not limited to, sugars, sugar alcohols, starches, ion exchangers, alumina, aluminium stearate, lecithin, serum proteins, such as human serum albumin, buffer substances such as phosphates, glycerine, sorbic acid, potassium sorbate, partial glyceride mixtures of saturated vegetable fatty acids, water, salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat, or a combination thereof.

The pharmaceutical compositions of this invention are preferably administered orally. In one embodiment, the composition may be combined with or sprinkled onto food for ingestion by a subject.

The composition of this invention may be administered with one or more additional active pharmaceutical ingredient, which may be incorporated into the composition or administered separately, either simultaneously or sequentially.

In a second aspect, the present invention provides a process for producing a granular composition comprising a pharmaceutically active agent, the process comprising:

(1) granulating a mixture comprising a pharmaceutically active agent and one or more pharmaceutically acceptable excipients using a first granulation liquid;
(2) drying the granules formed in (1);
(3) optionally, calibrating the size of the granules resulting from (2);
(4) granulating the granules resulting from (2) or (3) using a second granulation liquid;
(5) drying the granules formed in (4);
(6) coating the granules resulting from (5) using a coating liquid; and
(7) drying the coated granules formed in (6),
wherein at least 90% of the coated granules that are produced have a particle size of 90 μm or more and/or wherein at least 50% of the coated granules that are produced have a particle size of about 250 μm or more.

As will be appreciated by one skilled in the art there are numerous different types of laboratory equipment which are suitable for performing granulation, drying and/or coating of granules. For example, pieces of equipment suitable for granulation include a high shear granulator, a fluid bed dryer or a single plot system. Pieces of equipment suitable for drying granules include a fluid bed dryer, a continuous fluid bed, a single plot system and a tray dryer. Pieces of equipment suitable for coating granules include a fluid bed dryer, a coating machine and a vertical centrifugal coater. A skilled person will be familiar with these pieces of equipment, how they work and the various settings associated with such equipment.

In a high shear granulator, some of the parameters that can be changed to affect the granulation process are mixer speed, mixer current, product temperature, chopper speed and chopper current. Further, the length of time for which a particular parameter is set at a particular level can be varied and combinations of parameter settings can also be used, for example, combinations of speeds. A skilled person will be familiar with these parameters and will be fully aware of how to adjust these parameters on a high shear granulator.

In a fluid bed dryer, some of the parameters that can be changed to affect the granulation, drying and/or coating processes are inlet air temperature, product temperature, outlet air temperature, drying flux (also known as air flow speed), pump speed, pump pressure and type of nozzle. Further, the length of time for which a particular parameter is set at a particular level can be varied and combinations of parameter settings can also be used, for example, different combinations of temperatures and/or air flow. A skilled person will be familiar with these parameters and would be aware of how to adjust these parameters on a fluid bed dryer.

For the sake of clarity, some of the parameters referred to below have the following definitions:

“Inlet air temperature” means the temperature of the air that enters into the fluid bed dryer. It is measured continuously during the process with a thermometer attached to the entrance of the fluid bed dryer.

“Product temperature” (or “granule temperature”) means the temperature of the air and therefore, the product (or granules) in the fluid bed dryer. It is measured continuously during the process with a thermometer attached to the interior of the fluid bed dryer.

“Drying flux” or “flow” means the quantity of air that passes through a space of the fluid bed dryer per unit of time—m³/cm²/h. it is measured continuously by a flowmeter in the machine.

“Maximum flux capacity” means the maximum flux or flow that a dryer can provide.

“Fluid bed dryer total volume/minute” is a measure of the rate of introduction of a fluid, e.g., the granulation liquid, into the fluid bed dryer and depends on the size of the fluid bed dryer. For example, a rate of 10% of the fluid bed dryer total volume/minute corresponds to 5 L/minute for a 50 L fluid bed dryer and 100 L/minute for a 1000 L fluid bed dryer.

The mixture that is granulated in (1) may be any suitable mixture comprising a pharmaceutically active agent and one or more pharmaceutically acceptable excipients. The pharmaceutically active agent can be any pharmaceutically active agent of which it is desired to produce relatively large granules. In one embodiment, the pharmaceutically active agent is eslicarbazepine acetate.

In one embodiment, the mixture comprises between about 2% and about 98% by weight of the pharmaceutically active agent, such as eslicarbazepine acetate. In certain such embodiments, the mixture comprises at least about 3%, 4%, 5%, 6%, 7%, 8%, 9%, 10%, 11%, 12%, 13%, 14% or 15% of the pharmaceutically active agent, such as eslicarbazepine acetate, by weight. In certain such embodiments, the mixture comprises up to about 85%, 70%, 60%, 50%, 40%, 35%, 30%, 25%, 20% or 15% of the pharmaceutically active agent, such as eslicarbazepine acetate, by weight. In particular embodiments, the amount of the pharmaceutically active agent in the mixture by weight may be between about 5% and about 85%, between about 7% and about 70%, between about 10% and about 50%, between about 5% and about 25%, or between about 5% and about 15%.

The mixture may comprise a filler material. In one embodiment, the mixture comprises between about 2% and about 98% filler material by weight. In certain such embodiments, the mixture comprises at least about 5%, 10%, 15%, 20%, 25%, 30%, 35%, 40%, 45%, 50%, 55%, 60%, 65%, 70% or 75% filler material by weight. In certain such embodiments, the mixture comprises up to about 95%, 90%, 85%, 80% or 75% filler by weight. In certain embodiments, the mixture comprises between about 15% and about 95%, between about 30% and about 90%, between about 50% and about 80%, between about 60% and about 90%, or between about 70% and about 80% filler by weight.

The filler material may be selected from one or more of lactose, dibasic dihydrate calcium phosphate and isomalt. Preferably, the filler material is: lactose and dibasic dihydrate calcium phosphate; or isomalt and dibasic dihydrate calcium phosphate; or lactose and isomalt. More preferably, the filler material is lactose and dibasic dihydrate calcium phosphate.

When the filler material comprises lactose, the mixture preferably comprises between about 5% and about 90% lactose by weight. In certain embodiments, the mixture comprises at least about 10%, 15%, 20%, 25%, 30%, 35%, 40%, 45% or 50% lactose by weight. In certain embodiments, the mixture comprises up to about 85%, 80%, 75%, 70%, 65%, 60% or 55% lactose by weight. In certain embodiments, the amount of lactose in the mixture by weight may be between about 5% and about 80%, between about 15% and about 75%, between about 25% and about 60% or between about 40% and about 60%.

When the filler material comprises dibasic dihydrate calcium phosphate, the mixture preferably comprises between about 10% and about 50% dibasic dihydrate calcium phosphate by weight. More preferably, the amount of dibasic dihydrate calcium phosphate in the mixture by weight is between about 15% and about 50%, between about 10% and about 35%, between about 15% and about 30%, between about 15% and about 25% or between about 20% and about 25%.

When the filler material comprises isomalt, the mixture preferably comprises between about 5% and about 90% isomalt by weight. In certain embodiments, the mixture comprises at least about 10%, 15%, 20%, 25%, 30%, 35%, 40%, 45% or 50% isomalt by weight. In certain embodiments, the mixture comprises up to about 85%, 80%, 75%, 70%, 65%, 60% or 55% isomalt by weight. In certain embodiments, the amount of isomalt in the mixture by weight may be between about 5% and about 80%, between about 15% and about 75%, between about 25% and about 60% or between about 40% and about 60%.

In some embodiments, the mixture that is granulated in (1) further comprises a binder. The binder may be any suitable binder. In some instances, the binder may be selected from xanthan gum, HPMC, starch, sodium alginate and povidone. In one embodiment, the binder is povidone.

In certain embodiments, the first granulation liquid is an aqueous solution comprising a binder. The binder may be selected from xanthan gum, HPMC, starch, sodium alginate and povidone. In one embodiment, the first granulation liquid is an aqueous solution comprising povidone.

In some instances, the mixture that is granulated in (1) further comprises a colouring agent. In one embodiment, the first granulating liquid comprises a colouring agent.

Often, the mixture further comprises a sweetener and/or a flavouring agent. The first granulating liquid may comprise a sweetener and/or a flavouring agent.

The mixture may further comprise one or more additional active pharmaceutical ingredients.

Granulation in (1) may be carried out in any suitable granulator. In some embodiments, the granulation is carried out in a high shear granulator or a fluid bed dryer. In one embodiment, the granulation is carried out in a high shear granulator. In another embodiment, the granulation is carried out in a fluid bed dryer.

The first granulation liquid can be any suitable granulation liquid. In one embodiment, the first granulation liquid comprises water. Alternatively, the granulation liquid may comprise an alcohol, acetone or other organic solvents, or combinations thereof. As indicated above, the first granulation liquid be combined with a binder, a colouring agent, a sweetener and/or a flavouring agent.

In one embodiment, the first granulation liquid is added prior to commencement of granulation. Alternatively, the first granulation liquid may be added whilst granulation is taking place. In a particular embodiment, the rate of introduction of the first granulation liquid is increased over time.

When a high shear granulator is used to carry out granulation in (1), the granulator speed may be between about 50 rpm and about 500 rpm. In other embodiments, the granulator speed may be between about 50 rpm and about 400 rpm, between about 100 rpm and about 400 rpm, between about 150 rpm and about 350 rpm, between about 100 rpm and about 300 rpm, or between about 150 rpm and about 250 rpm.

When a high shear granulator is used to carry out granulation in (1), the chopper speed may be between about 50 rpm and about 5000 rpm. In other embodiments, the chopper speed may be between about 50 rpm and about 4000 rpm, between about 100 rpm and about 4000 rpm, between about 150 rpm and about 3500 rpm, or between about 200 rpm and about 3000 rpm.

In certain preferred embodiments, when a high shear granulator is used to carry out granulation in (1), the chopper speed may be between about 50 rpm and about 500 rpm. In other embodiments, the chopper speed may be between about 50 rpm and about 450 rpm, between about 100 rpm and about 400 rpm, between about 150 rpm and about 350 rpm, or between about 200 rpm and about 300 rpm.

When a fluid bed dryer is used to carry out granulation in (1), the rate of introduction of the first granulation liquid may be between about 0.02% and about 5% of the fluid bed dryer total volume/minute. In other embodiments, the rate of introduction of the first granulation liquid may be between about 0.02% and about 4%, between about 0.02% and about 3%, between about 0.02% and about 2%, or between about 0.02% and about 1% of the fluid bed dryer total volume/minute. The rate of introduction of the first granulation liquid may be controlled by the pump speed of the fluid bed dryer.

When a fluid bed dryer is used to carry out granulation in (1), air may be used to transport the first granulation liquid into the fluid bed dryer. The pressure of the air used to transport the granulation liquid into the fluid bed dryer may be between about 0.1 bar (10 kPa) and about 6 bar (600 kPa), between about 0.1 bar (10 kPa) and about 4 bar (400 kPa), between about 1 bar (100 kPa) and about 3 bar (300 kPa), or between about 1.5 bar (150 kPa) and about 2.5 bar (250 kPa).

When a fluid bed dryer is used to carry out granulation in (1), the air flow during granulation may be between about 10% and about 100% of the fluid bed dryer maximum flux capacity. In other embodiments, the air flow during granulation may be between about 20% and about 95%, between about 30% and about 90%, or between about 40% and about 90%, or between about 70% to about 80% of the fluid bed dryer maximum flux capacity.

In certain embodiments, when a fluid bed dryer is used to carry out granulation in (1), the air flow during granulation may be between about 20% and about 80%, between about 30% and about 70%, or between about 40% and about 60% of the fluid bed dryer maximum flux capacity.

In one embodiment, air flow during granulation may be increased in a stepwise manner over time.

When a fluid bed dryer is used to carry out granulation in (1), the temperature of the inlet air entering the fluid bed dryer during granulation may be between about 30° C. and about 80° C., between about 50° C. and about 80° C. or between about 60° C. and about 80° C.

The temperature of the mixture during granulation in (1) may be between about 10° C. and about 70° C. In other embodiments, the temperature of the mixture during granulation in (1) may be between about 20° C. and about 60° C., between about 25° C. and about 50° C. or between about 30° C. and about 50° C.

Drying in (2) may be carried out in any suitable dryer. In one embodiment, the drying is carried out in a fluid bed dryer. The drying may be continued until the relative humidity of the granules is about 6% or less, about 5% or less, about 4% or less, or about 3% or less. The relative humidity of the granules can be measured using a moisture balance or moisture analyser.

When a fluid bed dryer is used for drying in (2), the drying of the granules may take place at an inlet air and granule (product) temperature between about 40° C. and about 80° C., or between about 50° C. and about 80° C. Further, drying of the granules may take place at a drying flux of between about 20% and about 90% of the fluid bed dryer maximum flux capacity or at a drying flux of between about 20% and about 75% of the fluid bed dryer maximum flux capacity.

In certain embodiments, when a fluid bed dryer is used for drying in (2), the drying of the granules may take place at an inlet air and granule (product) temperature between about 50° C. and about 80° C., or between about 60° C. and about 80° C. Drying of the granules may take place at a drying flux of between about 20% and about 50% of the fluid bed dryer maximum flux capacity.

Calibrating in (3) may be used to ensure that the granules resulting from (2) are of a suitable size for granulation (4). If necessary, this may involve removing granules which are above a certain size or reducing the size of granules which are above a certain size. In effect, the calibrating step ensures that all the granules are below a certain size. This can be done in any suitable way, for example, using a sieve or a sieve battery. In some embodiments, a vibratory sieve or sieve battery can break up larger granules until they are small enough to pass through the holes of the sieve. The calibrating may comprise screening the granules resulting from (2) to ensure the particles have a particle size of about 2 mm or less, about 1.5 mm or less, or about 0.8 mm or less.

Granulation in (4) may be carried out in any suitable granulator. In one embodiment, the granulation is carried out in a fluid bed dryer.

In some embodiments, the granules that are granulated in (4) are granulated with a binder. The binder may be any suitable binder. In some instances, the binder may be selected from xanthan gum, HPMC, starch, sodium alginate and povidone. In one embodiment, the binder is povidone.

In certain embodiments, the second granulation liquid is an aqueous solution comprising a binder. The binder may be selected from xanthan gum, HPMC, starch, sodium alginate and povidone. In one embodiment, the second granulation liquid is an aqueous solution comprising povidone.

In some instances, the granules that are granulated in (4) are granulated with a colouring agent. In one embodiment, the second granulating liquid is combined with a colouring agent.

The second granulation liquid can be any suitable granulation liquid. In one embodiment, the second granulation liquid comprises water. Alternatively, the granulation liquid may comprise an alcohol, acetone or another organic solvent, or a combination thereof.

In one embodiment, the second granulation liquid is added prior to commencement of the granulation step. Alternatively, the second granulation liquid may be added whilst granulation is taking place. In a particular embodiment, the rate of introduction of the second granulation liquid is increased over time.

When a fluid bed dryer is used to carry out granulation in (4), the rate of introduction of the second granulation liquid may be between about 0.02% and about 5% of the fluid bed dryer total volume/minute. In other embodiments, the rate of introduction of the second granulation liquid may be between about 0.02% and about 4%, between about 0.02% and about 3%, between about 0.02% and about 2%, or between about 0.02% and about 1% of the fluid bed dryer total volume/minute. The rate of introduction of the second granulation liquid may be controlled by the pump speed of the fluid bed dryer.

When a fluid bed dryer is used to carry out granulation in (4), air may be used to transport the second granulation liquid into the fluid bed dryer. The pressure of the air used to transport the granulation liquid into the fluid bed dryer may be between about 0.1 bar (10 kPa) and about 6 bar (600 kPa), between about 0.1 bar (10 kPa) and about 4 bar (400 kPa), between about 1 bar (100 kPa) and about 3 bar (300 kPa), or between about 1.5 bar (150 kPa) and about 2.5 bar (250 kPa).

When a fluid bed dryer is used to carry out granulation in (4), the air flow during granulation may be between about 10% and about 100% of the fluid bed dryer maximum flux capacity. In other embodiments, the air flow during granulation may be between about 20% and about 95%, between about 30% and about 90%, or between about 40% and about 90% of the fluid bed dryer maximum flux capacity.

In certain embodiments, when a fluid bed dryer is used to carry out granulation in (4), the air flow during granulation may be between about 20% and about 80%, between about 30% and about 70%, or between about 40% and about 60% of the fluid bed dryer maximum flux capacity.

In one embodiment, air flow during granulation may be increased in a stepwise manner over time.

When a fluid bed dryer is used to carry out granulation in (4), the temperature of the inlet air entering the fluid bed dryer during granulation may be between about 30° C. and about 80° C., between about 50° C. and about 80° C., or between about 60° C. and about 80° C.

The temperature of the granules during granulation in (4) may be between about 10° C. and about 70° C. In other embodiments, the temperature of the granules during granulation in (4) may be between about 20° C. and about 60° C., between about 25° C. and about 50° C. or between about 30° C. and about 50° C.

Drying in (5) may be carried out in any suitable dryer. In one embodiment, the drying is carried out in a fluid bed dryer. The drying may be continued until the relative humidity of the granules is about 5% or less, about 4% or less, or about 3% or less.

When a fluid bed dryer is used for drying in (5), the drying of the granules may take place at an inlet air and granule (product) temperature between about 40° C. and about 80° C., or between about 50° C. and about 80° C. Further, drying of the granules may take place at a drying flux of between about 20% and about 90% of the fluid bed dryer maximum flux capacity or at a drying flux of between about 20% and about 75% of the fluid bed dryer maximum flux capacity.

In certain embodiments, when a fluid bed dryer is used for drying in (5), the drying of the granules may take place at an inlet air and granule (product) temperature between about 50° C. and about 80° C., or between about 60° C. and about 80° C. Drying of the granules may take place at a drying flux of between about 20% and about 50% of the fluid bed dryer maximum flux capacity.

Coating in (6) may be carried out using any suitable coating equipment. In one embodiment, the coating is carried out in a fluid bed dryer.

The coating liquid can be any suitable liquid containing components to form a coating on the granules. In one embodiment, the coating liquid is an aqueous solution. Alternatively, the coating liquid may comprise an alcohol, acetone or another organic solvent, or combinations thereof.

When a fluid bed dryer is used to carry out (6), the rate of introduction of the coating liquid may be between about 0.02% and about 5% of the fluid bed dryer total volume/minute. In other embodiments, the rate of introduction of the coating liquid may be between about 0.02% and about 4%, between about 0.02% and about 3%, between about 0.02% and about 2%, or between about 0.02% and about 1% of the fluid bed dryer total volume/minute.

When a fluid bed dryer is used to carry out (6), air may be used to transport the coating liquid into the fluid bed dryer. The pressure of the air used to transport the coating liquid into the fluid bed dryer may be between about 0.1 bar (10 kPa) and about 6 bar (600 kPa).

When a fluid bed dryer is used to carry out (6), the temperature of the inlet air entering the fluid bed dryer during coating may be between about 30° C. and about 80° C.

The temperature of the granules in (6) may be between about 10° C. and about 70° C. In other embodiments, the temperature of the granules may be between about 20° C. and about 60° C., between about 25° C. and about 50° C. or between about 30° C. and about 50° C.

In some embodiments, the granules that are coated in (6) are coated with a colouring agent. In one embodiment, the coating liquid comprises a colouring agent. When the coating liquid comprises a colouring liquid the granules can be coated such that any unpleasant flavour, for example due to the taste of the excipients or eslicarbazepine acetate, is masked until the granules have been swallowed.

Drying in (7) may be carried out in any suitable dryer. In one embodiment, the drying is carried out in a fluid bed dryer. The drying may be continued until the relative humidity of the granules is about 5% or less, about 4% or less, or about 3% or less.

When a fluid bed dryer is used for drying in (7), the drying of the granules may take place at an inlet air and granule (product) temperature between about 40° C. and about 80° C., or between about 50° C. and about 80° C. Further, drying of the granules may take place at a drying flux of between about 20% and about 90% of the fluid bed dryer maximum flux capacity or at a drying flux of between about 20% and about 75% of the fluid bed dryer maximum flux capacity.

In certain embodiments, when a fluid bed dryer is used for drying in (7), the drying of the granules may take place at an inlet air and granule (product) temperature between about 50° C. and about 80° C., or between about 60° C. and about 80° C. Drying of the granules may take place at a drying flux of between about 20% and about 50% of the fluid bed dryer maximum flux capacity.

It will be appreciated by one skilled in the art that although (1) and (2), (4) and (5), and (6) and (7) have been presented as being separate, if these steps are carried out in the same apparatus or piece of equipment, they may take place at the same time or there may be an overlap in the timings of when these steps are being carried out. For example, granulation may still be taking place whilst the granules are in the process of being dried. A short period of granulation may take place, followed by a period of granulation and drying, followed by a period of drying. In this way, some of the parameters associated with granulation may also be applicable to drying and vice versa.

In another aspect, the invention provides a composition which is obtainable by the process described above and/or a composition which is produced by the process described above.

Since the process described above can be used to produce the compositions described in the first aspect of the invention, a skilled person will appreciate that many of the limitations described for the compositions are equally applicable to the process, for example, the limitations relating to the size of the granules that are produced by the method and the identity and quantities of excipients, filler material and binders.

In a further aspect, the invention provides any of the compositions described above for use in therapy.

In yet another aspect, the invention provides any of the compositions described above, in which the pharmaceutically active agent is eslicarbazepine acetate, for use in the treatment or prevention of a disorder selected from epilepsy, neuropathic pain, migraine, fibromyalgia and an affective disorder.

The invention also provides the use of any of the compositions described above, in which the pharmaceutically active agent is eslicarbazepine acetate, in the manufacture of a medicament for the treatment or prevention of a disorder selected from epilepsy, neuropathic pain, migraine, fibromyalgia and an affective disorder.

In certain embodiments, neuropathic pain is selected from trigeminal neuralgia, phantom pain, diabetic neuropathy and postherpetic neuralgia.

In certain embodiments, the affective disorder is selected from bipolar disorder, depression, pre-menstrual dysphoric disorder, post-partum depression, post-menopausal depression, anorexia nervosa, bulimia nervosa, or neurodegeneration-related depressive symptoms, unstable bipolar disorder with rapid fluctuations (rapid cycles), manic-depressive disorder, acute mania, a mood episode, a manic episode, and a hypomanic episode.

The invention also provides a method of treating or preventing a disorder, the method comprising the administration of an effective amount of the composition described above, in which the pharmaceutically active agent is eslicarbazepine acetate, to a subject in need thereof, wherein the disorder is selected from epilepsy, neuropathic pain, migraine, fibromyalgia and an affective disorder.

In one embodiment, the subject is human.

The compositions may be administered with other active pharmaceutical ingredient(s). Such combination therapy includes simultaneous and sequential administration of the composition of the invention with the other active pharmaceutical ingredient(s).

### DESCRIPTION OF FIGURES

FIG. 1 shows the relationships between the distribution coefficient and the tristimulus values for colour determination.

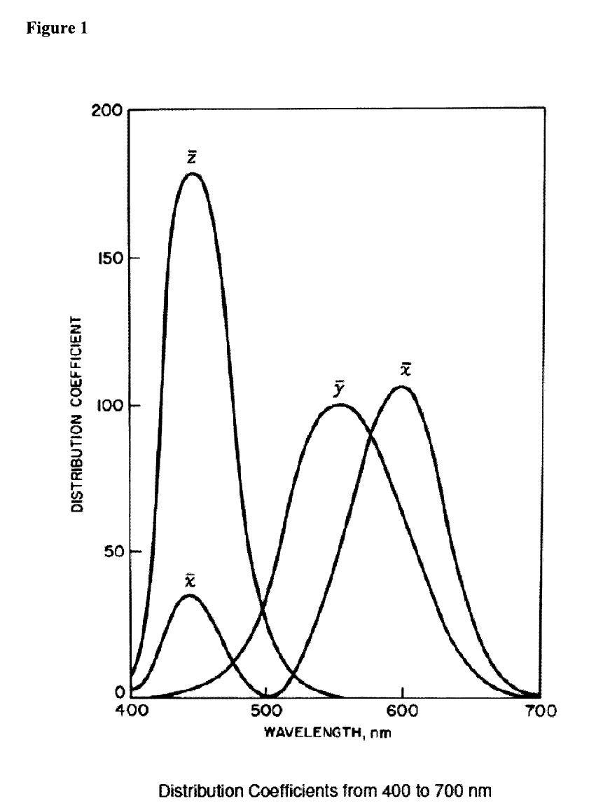

FIG. 2 shows the granule distribution of the API pilot batches of Table 3.

FIG. 3 shows the granule size distribution for two batches according to the invention (Batches 18 and 19) compared to the granule size distribution of a batch (Batch 20) which is representative of a composition that may be used in the production of tablets.

FIG. 4 shows the equipment for measuring Apparent Volume of a granule composition.

FIG. 5 shows the dimensions (in cm) of a funnel for measuring the flow of a granule composition.

### DETAILED DESCRIPTION OF THE INVENTION

The invention will now be described in more detail, by way of example only. The following examples are not intended to be limiting.

### Example 1

### Development of Granule Formulation

### Experimental Part

### Equipment

The formulation work was performed the following equipment:
Balance Mettler Toledo model PM 1200, code 5006;
Balance AND GX-1000, code 5033;
Ika mixer RW20, code 5002;
Laboratory Erweka oscillating granulator type FGS with a 1.6 mm sieve coupled to
Erweka rotor type KU1, code 5007;
Laboratory V blender coupled to Erweka rotor type AR402, code 5015;
Hearson dryer;
Silase 50 L biconic blender, code 5031;
Diosna P-VAC 60 mixer/granulator, code 5026; and
Diosna CAP 50 fluid bed dryer, code 5025.

The following equipment was used to test the samples:
Balance Mettler Toledo, model AG 245, code 4122;
Vibrating sieve battery, code 4008;
Varian VK7025 dissolution apparatus coupled to a UV/Vis spectrophotometer Cary 50 tablet through a peristaltic pump Varian VK800, code 5024;
Waters Alliance HPLC, model 2695, with a diode array detector model 2996, code 4040; and
Waters Alliance HPLC, model 2695, with a diode array detector model 2996, code 5020.

### Parameters and Methods

### Pilot Batches

The batches were prepared using one of the following procedures:

Procedure 1:
1. Mix components in a fluid bed dryer;
2. Add the granulation liquid and granulate;
3. Dry until the granule humidity is below 3%.

Procedure 2:
1. Mix eslicarbazepine acetate and the main excipient in a 50 L biconic blender;
2. Transfer the mix to a fluid bed dryer;
3. Add the granulation liquid (with saccharin) and granulate;
4. Dry until the granule humidity is below 3%;
5. Repeat steps 3 and 4 (2″ granulation);
6. Add coating solution (with flavour);
7. Dry until the granule humidity is below 3%.

Procedure 3:
1. Mix eslicarbazepine acetate and the main excipient in a Diosna mixer/granulator;
2. Add the granulation liquid and granulate;
3. Transfer the mix to a fluid bed dryer and dry until the granule humidity is below 3%;
4. Add the granulation liquid and granulate;
5. Dry until the granule humidity is below 3%;
6. Add coating solution;
7. Dry until the granule humidity is below 3%.

### Granulometric Distribuition

The granulometric distribution was performed using a sieve battery following USP procedure <786—Particle size distribution estimation by analytical sieving>. See United States Pharmacopoeia 31, The National Formulary 26, 2008, Rockville.

### Granule API Assay and Dissolution

An eslicarbazepine acetate assay was performed by HPLC and all batches gave satisfactory results i.e., an API assay of 95-105%. In some embodiments, the compositions of the batches gave an assay value of at least about 50%, 60%, 70%, 80%, 85%, 90%, 95%, 96%, 97%, 98% or 99%. The dissolution was performed using a rotating paddle apparatus at 75 rpm and 100 rpm, and quantification was performed using HPLC (See <711—Dissolution>. See United States Pharmacopoeia 31, The National Formulary 26, 2008, Rockville). All batches showed satisfactory results i.e., dissolution of >85% after 45 minutes. In some embodiments, the compositions of the batches gave a dissolution value of at least about 50%, 60%, 70%, 80%, 85%, 90% or 95%.

Colour homogeneity was assessed visually.

### Raw Materials

TABLE 1
Ingredients used in the manufacturing processes

| Ingredient | Function |
|---|---|
| eslicarbazepine acetate | API |
| Manukol ® LKX (Sodium Alginate) | Filler/binder |
| Avicel PH102 (microcrystalline | Filler/disintegrant |
| cellulose) | |
| Starch 1500 (pregelatinized maize | Filler/binder/disintegrant |
| starch) | |
| Xanthan Gum | Filler |
| HPMC | Binder |
| PEG 6000 | Binder |
| Povidone K-30 | Binder |
| Emcompress ® (Dihydrate & Dibasic | Filler |
| Calcium Phosphate) | |
| Lactose 200M | Filler |
| GalenIQ ® 800 (isomalt) | Filler |
| GalenIQ ® 801 (isomalt) | Filler |
| Eudragit ® RL PO (Poly(ethyl | Binder |
| acrylate-co-methyl methacrylate-co- | |
| trimethylammonioethyl methacrylate | |
| chloride) | |
| Saccharin | Sweetening agent |
| Strawberry flavour | Flavour |
| Opadry II RED | Colouring agent |
| AquaPolish D RED | Colouring agent |
| Lactose 80M | Filler |

### Batches

TABLE 2
Batches produced during development

| Size | API/Placebo | Filler | Binder | Other excipients | Batch |
|---|---|---|---|---|---|
| Pilot | API | Lactose 200M | Povidone K-30 | Opadry ® II RED 31K (3.20%) | Batch 1 |
| | | Lactose 200M (32.5%) + | Povidone K-30 | Opadry ® II RED 31K (6.25%); Saccharin | Batch 2 |
| | | Emcompress ® (40.8%) | | (0.3%); Strawberry flavour (0.15%) | |
| | | Emcompress ®* | Povidone K-30 | Opadry ® II RED 31K (6.25%); Saccharin | Batch 3; Batch 4 |
| | | | | (0.3%); Strawberry flavour (0.15%) | |
| | | | | Saccharin (0.3%) | Batch 5; Batch 6; |
| | | | | | Batch 7; Batch 8 |
| | | | | Crospovidone (5%) | Batch 9 |
| | | Lactose 200M (36.7%) + | Povidone K-30 | Saccharin (0.3%) | Batch 10 |
| | | Emcompress ® (36.7%) | | | |
| | | Lactose 200M (53.0%) + | Povidone K-30 | Opadry ® II RED 31K (6.25%); Saccharin | Batch 11; |
| | | Emcompress ® (22.3%) | | (0.3%); Strawberry flavour (0.15%) | Batch 14; |
| | | Lactose 200M (53.0%) + | Povidone K-30 | Opadry ® II RED 31B (6.25%); Saccharin | Batch 13 |
| | | Emcompress ® (22.3%) | | (0.3%); Strawberry flavour (0.15%) | |
| Pilot | API | GalenIQ ® 800 (51.0%) + | Povidone K-30 | Opadry ® II RED 31K (6.25%); Saccharin | Batch 12 |
| | | Emcompress ® (22.3%) | | (0.3%); Strawberry flavour (0.15%) | |
| | | GalenIQ ® 801 (51.0%) + | Povidone K-30 | Opadry ® II RED 31K (6.25%); Saccharin | Batch 15 |
| | | Emcompress ® (22.3%) | | (0.3%); Strawberry flavour (0.15%) | |
| | | GalenIQ ® 801 (51.0%) + | Povidone K-30 | Opadry ® II RED 03B (6.25%); Saccharin | Batch 16 |
| | | Emcompress ® (22.3%) | | (0.3%); Strawberry flavour (0.15%) | |
| | | GalenIQ ® 801 (51.0%) + | Povidone K-30 | AquaPolish D RED (6.25%); Saccharin | Batch 17 |
| | | Emcompress ® (22.3%) | | (0.3%); Strawberry flavour (0.15%) | |

### Data Evaluation

### Pilot Batches

TABLE 3
API pilot batches d10, d50 and d90

| Batch | d10 (mm) | d50 (mm) | d90 (mm) |
|---|---|---|---|
| Batch 1 | 0.09 | 0.18 | 0.50 |
| Batch 2 | 0.18 | 0.30 | 0.30 |
| Batch 3 | 0.18 | 0.30 | 0.50 |
| Batch 4 | 0.18 | 0.30 | 0.50 |
| Batch 5 | 0.18 | 0.50 | 1.60 |
| Batch 6 | 0.18 | 0.50 | 0.71 |
| Batch 7 | 0.18 | 0.50 | 1.60 |
| Batch 8 | 0.18 | 0.30 | 0.50 |
| Batch 9 | 0.09 | 0.30 | 0.50 |
| Batch 10 | 0.18 | 0.30 | 0.50 |
| Batch 11 | 0.18 | 0.18 | 0.30 |
| Batch 12 | 0.30 | 0.50 | 0.71 |

From the results presented in table 3, all batches presented adequate granulometry. The batches containing Emcompress® and galenIQ® 800 present higher d10 and d90 values.

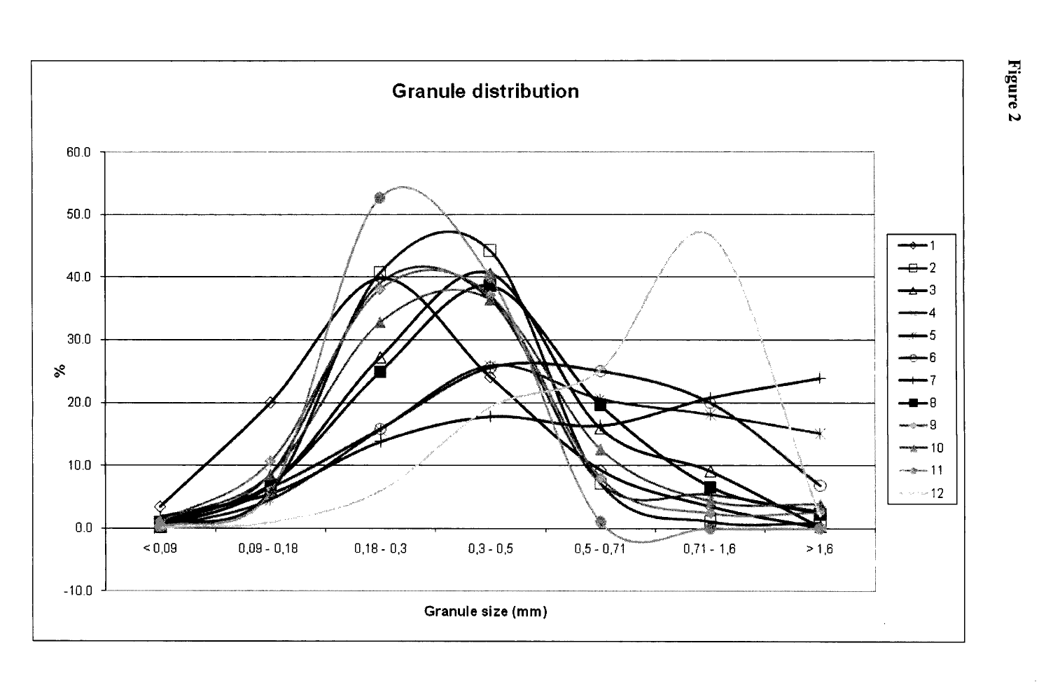

From the granule distribution results in FIG. 2, it can be said that not all granulates have the similar properties. The lower particle size granulate is clearly Batch 1, which is the only one using lactose alone as a main excipient.

Batch 5, Batch 6, Batch 7 and Batch 8 present a more dispersed granule size, which may be due to the initial wetting before granulation.

The higher particle size granulate is clearly Batch 12, which is the only one using galenIQ® 800 alone as a main excipient.

All other batches presented similar granule distribution.

TABLE 4
Pilot batches

| | | Other | | | |
|---|---|---|---|---|---|
| Filler | Binder | excipients | Batch | Manufacture procedure/conditions | Results |
| Lactose | Povidone | Opadry ® II | Batch | Procedure 2. | Colour |
| 200M | K-30 | RED 31K | 1 | Granulation 1: | homogeneity: |
| | | (3.20%) | | Flux: 570 m³/h/cm² | Not |
| | | | | Initial pump speed: 0 rpm | homogeneous |
| | | | | Granulation liquid: povidone + water | Assay: N/A |
| | | | | Atomisation pressure: 3 BAR (300 | Dissolution: |
| | | | | kPa) | N/A |
| | | | | Inlet air temperature: 77° C. | |
| | | | | Pump speed: 50 rpm continuous | |
| | | | | Drying flux: 500 m³/h/cm² | |
| | | | | Drying temperature: 77° C. | |
| | | | | Granulation 2: | |
| | | | | Flux: 520 m³/h/cm² | |
| | | | | Initial pump speed: 0 rpm | |
| | | | | Granulation liquid: povidone + water | |
| | | | | Atomisation pressure: 3 BAR (300 | |
| | | | | kPa) | |
| | | | | Inlet air temperature: 77° C. | |
| | | | | Pump speed: 45 rpm continuous | |
| | | | | Drying flux: 500 m³/h/cm² | |
| | | | | Drying temperature: 77° C. | |
| | | | | Coating: | |
| | | | | Flux: 400 m³/h/cm² | |
| | | | | Atomisation pressure: 2 BAR (200 | |
| | | | | kPa) | |
| | | | | Inlet air temperature: 55° C. | |
| | | | | Pump speed: 4 rpm continuous | |
| | | | | Drying flux: 250 m³/h/cm² | |
| | | | | Drying temperature: 77° C. | |
| Lactose | Povidone | Opadry ® II | Batch | Procedure 2. | Colour |
| 200M | K-30 | RED 31K | 2 | Granulation 1: | homogeneity: |
| (32.5%) + | | (6.25%) | | Flux: 565 m³/h/cm² | Not homogeneous |
| Emcompress ® | | Saccharin | | Initial pump speed: 0 rpm | Assay: N/A |
| (40.8%) | | (0.3%); | | Granulation liquid: povidone + | Dissolution: N/A |
| | | Strawberry | | Saccharin + water | |
| | | flavour | | Atomisation pressure: 2 BAR | |
| | | (0.15%) | | (200 kPa) | |
| | | | | Inlet air temperature: 77° C. | |
| | | | | Pump speed: 50 rpm | |
| | | | | continuous | |
| | | | | Drying flux: 300 m³/h/cm² | |
| | | | | Drying temperature: 77° C. | |
| | | | | Granulation 2: | |
| | | | | Flux: 550 m³/h/cm² | |
| | | | | Initial pump speed: 0 rpm | |
| | | | | Granulation liquid: povidone + | |
| | | | | water | |
| | | | | Atomisation pressure: 2 BAR | |
| | | | | (200 kPa) | |
| | | | | Inlet air temperature: 77° C. | |
| | | | | Pump speed: 40 rpm | |
| | | | | continuous | |
| | | | | Drying flux: 300 m³/h/cm² | |
| | | | | Drying temperature: 77° C. | |
| | | | | Coating: | |
| | | | | Flux: 400 m³/h/cm² | |
| | | | | Atomisation pressure: 2 BAR | |
| | | | | (200 kPa) | |
| | | | | Inlet air temperature: 55° C. | |
| | | | | Pump speed: 4 rpm continuous | |
| | | | | Drying flux: 250 m³/h/cm² | |
| | | | | Drying temperature: 77° C. | |
| Emcompress ®* | Povidone | Opadry ® II | Batch | Procedure 2. | Colour |
| | K-30 | RED 31K | 3 | Granulation 1: | homogeneity: |
| | | (6.25%); | | Flux: 540 m³/h/cm² | homogeneous |
| | | Saccharin | | Initial pump speed: 0 rpm | Assay: 48.9% |
| | | (0.3%); | | Granulation liquid: povidone + | Dissolution: N/A |
| | | Strawberry | | Saccharin + coating agent + | |
| | | flavour | | water | |
| | | (0.15%) | | Atomisation pressure: 2 BAR | |
| | | | | (200 kPa) | |
| | | | | Inlet air temperature: 77° C. | |
| | | | | Pump speed: 45 rpm | |
| | | | | continuous | |
| | | | | Drying flux: 300 m³/h/cm² | |
| | | | | Drying temperature: 77° C. | |
| | | | | Granulation 2: | |
| | | | | Flux: 500 m³/h/cm² | |
| | | | | Initial pump speed: 0 rpm | |
| | | | | Granulation liquid: povidone + | |
| | | | | coating agent + water | |
| | | | | Atomisation pressure: 2 BAR | |
| | | | | (200 kPa) | |
| | | | | Inlet air temperature: 77° C. | |
| | | | | Pump speed: 35 rpm | |
| | | | | continuous | |
| | | | | Drying flux: 300 m³/h/cm² | |
| | | | | Drying temperature: 77° C. | |
| | | | | Coating: | |
| | | | | Flux: 250 m³/h/cm² | |
| | | | | Atomisation pressure: 2 BAR | |
| | | | | (200 kPa) | |
| | | | | Inlet air temperature: 52° C. | |
| | | | | Pump speed: 4 rpm continuous | |
| | | | | Drying flux: 250 m³/h/cm² | |
| | | | | Drying temperature: 52° C. | |
| Emcompress ®* | Povidone | Opadry ® II | Batch | Procedure 2. | Colour |
| | K-30 | RED 31K | 4 | Granulation 1: | homogeneity: |
| | | (6.25%); | | Flux: 110, 220, 330, 440, 550 | homogeneous |
| | | Saccharin | | m³/h/cm² steps | Assay: 57.1% |
| | | (0.3%); | | Initial pump speed: 0 rpm | Dissolution: N/A |
| | | Strawberry | | Granulation liquid: povidone + | |
| | | flavour | | Saccharin + coating agent + | |
| | | (0.15%) | | water | |
| | | | | Atomisation pressure: 2 BAR | |
| | | | | (200 kPa) | |
| | | | | Inlet air temperature: 77° C. | |
| | | | | Pump speed: 9, 18, 27, 36, 45 rpm | |
| | | | | steps | |
| | | | | Drying flux: 350 m³/h/cm² | |
| | | | | Drying temperature: 77° C. | |
| | | | | Granulation 2: | |
| | | | | Flux: 110, 220, 330, 440, 550 | |
| | | | | m³/h/cm² steps | |
| | | | | Initial pump speed: 0 rpm | |
| | | | | Granulation liquid: povidone + | |
| | | | | coating agent + water | |
| | | | | Atomisation pressure: 2 BAR | |
| | | | | (200 kPa) | |
| | | | | Inlet air temperature: 77° C. | |
| | | | | Pump speed: 9, 18, 27, 36, 45 rpm | |
| | | | | steps | |
| | | | | Drying flux: 350 m³/h/cm² | |
| | | | | Drying temperature: 77° C. | |
| | | | | Coating: | |
| | | | | Flux: 250 m³/h/cm² | |
| | | | | Atomisation pressure: 2 BAR | |
| | | | | (200 kPa) | |
| | | | | Inlet air temperature: 52° C. | |
| | | | | Pump speed: 4 rpm continuous | |
| | | | | Drying flux: 250 m³/h/cm² | |
| | | | | Drying temperature: 52° C. | |
| Emcompress ®* | Povidone | Saccharin | Batch | Procedure 2. | Colour |
| | K-30 | (0.3%) | 5 | Granulation 1: | homogeneity: N/A |
| | | | | Flux: 540 m³/h/cm² | Assay: 79.8% |
| | | | | Initial pump speed: 7 rpm | Dissolution |
| | | | | Granulation liquid: povidone + | (30 minutes, |
| | | | | Saccharin + water | 100 rpm): 52.1% |
| | | | | Atomisation pressure: 2 BAR | |
| | | | | (200 kPa) | |
| | | | | Inlet air temperature: 77° C. | |
| | | | | Pump speed: 45 rpm continuous | |
| | | | | Drying flux: 300 m³/h/cm² | |
| | | | | Drying temperature: 77° C. | |
| | | | | Granulation 2: | |
| | | | | Flux: 500 m³/h/cm² | |
| | | | | Initial pump speed: 7 rpm | |
| | | | | Granulation liquid: povidone + | |
| | | | | water | |
| | | | | Atomisation pressure: 2 BAR | |
| | | | | (200 kPa) | |
| | | | | Inlet air temperature: 77° C. | |
| | | | | Pump speed: 35 rpm continuous | |
| | | | | Drying flux: 300 m³/h/cm² | |
| | | | | Drying temperature: 77° C. | |
| Emcompress ®* | Povidone | Saccharin | Batch | Procedure 2. | Colour |
| | K-30 | (0.3%) | 6 | Granulation 1: | homogeneity: N/A |
| | | | | Flux: 110, 220, 330, 440, 550 | Assay: 81.9% |
| | | | | m³/h/cm² steps | Dissolution |
| | | | | Initial pump speed: 9 rpm | (30 minutes, |
| | | | | Granulation liquid: povidone + | 100 rpm): 47.7% |
| | | | | Saccharin + water | |
| | | | | Atomisation pressure: 2 BAR | |
| | | | | (200 kPa) | |
| | | | | Inlet air temperature: 77° C. | |
| | | | | Pump speed: 9, 18, 27, 36, 45 rpm | |
| | | | | steps | |
| | | | | Drying flux: 350 m³/h/cm² | |
| | | | | Drying temperature: 77° C. | |
| | | | | Granulation 2: | |
| | | | | Flux: 110, 220, 330, 440, 550 | |
| | | | | m³/h/cm² steps | |
| | | | | Initial pump speed: 9 rpm | |
| | | | | Granulation liquid: povidone + | |
| | | | | water | |
| | | | | Atomisation pressure: 2 BAR | |
| | | | | (200 kPa) | |
| | | | | Inlet air temperature: 77° C. | |
| | | | | Pump speed: 9, 18, 27, 36, 45 rpm | |
| | | | | steps | |
| | | | | Drying flux: 350 m³/h/cm² | |
| | | | | Drying temperature: 77° C. | |
| Emcompress ®* | Povidone | Saccharin | Batch | Procedure 3. | Colour |
| | K-30 | (0.3%) | 7 | Granulation 1: | homogeneity: N/A |
| | | | | Granulator speed: 200 rpm | Assay: 97.1% |
| | | | | Chopper speed: 250 rpm | Dissolution |
| | | | | Granulation liquid: 500 ml | (30 minutes, |
| | | | | Granulation liquid: povidone + | 100 rpm): 62.75% |
| | | | | Saccharin + water | |
| | | | | Drying flux: 250 m³/h/cm² | |
| | | | | Drying temperature: 77° C. | |
| | | | | Granulation 2: | |
| | | | | Flux: 110, 220, 330, 440, 550 | |
| | | | | m³/h/cm² steps | |
| | | | | Initial pump speed: 9 rpm | |
| | | | | Granulation liquid: povidone + | |
| | | | | water | |
| | | | | Atomisation pressure: 2 BAR | |
| | | | | (200 kPa) | |
| | | | | Inlet air temperature: 77° C. | |
| | | | | Pump speed: 9, 18, 27, 36, 45 rpm | |
| | | | | steps | |
| | | | | Drying flux: 350 m³/h/cm² | |
| | | | | Drying temperature: 77° C. | |
| Emcompress ®* | Povidone | Saccharin | Batch | Procedure 2. | Colour |
| | K-30 | (0.3%) | 8 | Granulation 1: | homogeneity: N/A |
| | | | | Flux: 110, 220, 330, 440, 550 | Assay: 87.7% |
| | | | | m³/h/cm² step | Dissolution |
| | | | | Initial pump speed: 15 rpm | (30 minutes, |
| | | | | Granulation liquid: povidone + | 100 rpm): 75.9 |
| | | | | Saccharin + water | |
| | | | | Atomisation pressure: 2 BAR | |
| | | | | (200 kPa) | |
| | | | | Inlet air temperature: 77° C. | |
| | | | | Pump speed: 9, 18, 27, 36, 45 rpm | |
| | | | | steps | |
| | | | | Drying flux: 350 m³/h/cm² | |
| | | | | Drying temperature: 77° C. | |
| | | | | Granulation 2: | |
| | | | | Flux: 110, 220, 330, 440, 550 | |
| | | | | m³/h/cm² steps | |
| | | | | Initial pump speed: 15 rpm | |
| | | | | Granulation liquid: povidone + | |
| | | | | water | |
| | | | | Atomisation pressure: 2 BAR | |
| | | | | (200 kPa) | |
| | | | | Inlet air temperature: 77° C. | |
| | | | | Pump speed: 9, 18, 27, 36, 45 rpm | |
| | | | | steps | |
| | | | | Drying flux: 350 m³/h/cm² | |
| | | | | Drying temperature: 77° C. | |
| Emcompress ®* | Povidone | Crospovidone | Batch | Procedure 3. | Colour |
| | K-30 | (5%) | 9 | Granulation 1: | homogeneity: N/A |
| | | | | Granulator speed: 200 rpm | Assay: N/A |
| | | | | Chopper speed: 250 rpm | Dissolution |
| | | | | Granulation liquid: 400 ml | (30 minutes, |
| | | | | Granulation liquid: povidone + | 100 rpm): 71.8% |
| | | | | water | |
| | | | | Drying flux: 250 m³/h/cm² | |
| | | | | Drying temperature: 77° C. | |
| | | | | Granulation 2: | |
| | | | | Flux: 550 m³/h/cm² | |
| | | | | Initial pump speed: 7 rpm | |
| | | | | Granulation liquid: povidone + | |
| | | | | water | |
| | | | | Atomisation pressure: 2 BAR | |
| | | | | (200 kPa) | |
| | | | | Inlet air temperature: 77° C. | |
| | | | | Pump speed: 30-35 rpm | |
| | | | | Drying flux: 200 m³/h/cm² | |
| | | | | Drying temperature: 77° C. | |
| Lactose 200M | Povidone | Saccharin | Batch | Procedure 3. | Colour |
| (36.7%) + | K-30 | (0.3%) | 10 | Granulation 1: | homogeneity: N/A |
| Emcompress ® | | | | Granulator speed: 200 rpm | Assay: 100.4% |
| (36.7%) | | | | Chopper speed: 250 rpm | Dissolution |
| | | | | Granulation liquid: 400 ml | (30 minutes, |
| | | | | Granulation liquid: povidone + | 100 rpm): 88.6% |
| | | | | Saccharin + water | |
| | | | | Drying flux: 250 m³/h/cm² | |
| | | | | Drying temperature: 77° C. | |
| | | | | Granulation 2: | |
| | | | | Flux: 550 m³/h/cm² | |
| | | | | Initial pump speed: 7 rpm | |
| | | | | Granulation liquid: povidone + | |
| | | | | water | |
| | | | | Atomisation pressure: 2 BAR | |
| | | | | (200 kPa) | |
| | | | | Inlet air temperature: 77° C. | |
| | | | | Pump speed: 7, 14, 21, 28, 35 rpm | |
| | | | | steps | |
| | | | | Drying flux: 200 m³/h/cm² | |
| | | | | Drying temperature: 77° C. | |
| Lactose 200M | Povidone | Opadry ® II | Batch | Procedure 3. | Colour |
| (53.0%) + | K-30 | RED 31K | 11 | Granulation 1: | homogeneity: |
| Emcompress ® | | (6.25%); | | Granulator speed: 200 rpm | Homogeneous |
| (22.3%) | | Saccharin | | Chopper speed: 250 rpm | Assay: 94.5% |
| | | (0.3%); | | Granulation liquid: 700 ml | Dissolution: |
| | | Strawberry | | Granulation liquid: povidone + | 30 minutes, |
| | | flavour | | Saccharin + coating agent + | 75 rpm: 92.8% |
| | | (0.15%) | | water | 30 minutes, |
| | | | | Drying flux: 250 m³/h/cm² | 100 rpm: 92.6% |
| | | | | Drying temperature: 77° C. | |
| | | | | Granulation 2: | |
| | | | | Flux: 550 m³/h/cm² | |
| | | | | Initial pump speed: 7 rpm | |
| | | | | Granulation liquid: povidone + | |
| | | | | coating agent + water | |
| | | | | Atomisation pressure: 2 BAR | |
| | | | | (200 kPa) | |
| | | | | Inlet air temperature: 77° C. | |
| | | | | Pump speed: 35 rpm continuous | |
| | | | | Drying flux: 200 m³/h/cm² | |
| | | | | Drying temperature: 77° C. | |
| | | | | Coating: | |
| | | | | Flux: 250 m³/h/cm² | |
| | | | | Atomisation pressure: 2 BAR | |
| | | | | (200 kPa) | |
| | | | | Inlet air temperature: 52° C. | |
| | | | | Pump speed: 4 rpm continuous | |
| | | | | Drying flux: 250 m³/h/cm² | |
| | | | | Drying temperature: 52° C. | |
| Lactose 200M | Povidone | Opadry ® II | Batch | Procedure 3. | Colour |
| (53.0%) + | K-30 | RED 03B | 13 | Granulation 1: | homogeneity: |
| Emcompress ® | | (6.25%); | | Granulator speed: 200 rpm | Not Homogeneous |
| (22.3%) | | Saccharin | | Chopper speed: 250 rpm | Assay: N/A |
| | | (0.3%); | | Granulation liquid: 700 ml | Dissolution: N/A |
| | | Strawberry | | Granulation liquid: povidone + | |
| | | flavour | | Saccharin + coating agent + | |
| | | (0.15%) | | water | |
| | | | | Drying flux: 250 m³/h/cm² | |
| | | | | Drying temperature: 77° C. | |
| | | | | Granulation 2: | |
| | | | | Flux: 550 m³/h/cm² | |
| | | | | Initial pump speed: 7 rpm | |
| | | | | Granulation liquid: povidone + | |
| | | | | coating agent + water | |
| | | | | Atomisation pressure: 2 BAR | |
| | | | | (200 kPa) | |
| | | | | Inlet air temperature: 77° C. | |
| | | | | Pump speed: 35 rpm continuous | |
| | | | | Drying flux: 200 m³/h/cm² | |
| | | | | Drying temperature: 77° C. | |
| | | | | Coating: | |
| | | | | Flux: 250 m³/h/cm² | |
| | | | | Atomisation pressure: 2 BAR | |
| | | | | (200 kPa) | |
| | | | | Inlet air temperature: 52° C. | |
| | | | | Pump speed: 4 rpm continuous | |
| | | | | Drying flux: 250 m³/h/cm² | |
| | | | | Drying temperature: 52° C. | |
| Lactose 200M | Povidone | Opadry ® II | Batch | Procedure 3. | Colour |
| (53.0%) + | K-30 | RED 31K | 14 | Granulation 1: | homogeneity: |
| Emcompress ® | | (6.25%); | | Granulator speed: 200 rpm | Homogeneous |
| (22.3%) | | Saccharin | | Chopper speed: 250 rpm | Assay: N/A |
| | | (0.3%); | | Granulation liquid: 400 ml | Dissolution: |
| | | Strawberry | | Granulation liquid: povidone + | 30 minutes, |
| | | flavour | | Saccharin + coating agent + | 100 rpm: 94.2% |
| | | (0.15%) | | water | |
| | | | | Drying flux: 250 m³/h/cm² | |
| | | | | Drying temperature: 66° C. | |
| | | | | Calibration: After drying/ | |
| | | | | 1.2 mm screen/350 RPM | |
| | | | | Granulation 2: | |
| | | | | Flux: 550 m³/h/cm² | |
| | | | | Initial pump speed: 5 rpm | |
| | | | | Granulation liquid: povidone + | |
| | | | | coating agent + water | |
| | | | | Atomisation pressure: 2 BAR | |
| | | | | (200 kPa) | |
| | | | | Inlet air temperature: 66° C. | |
| | | | | Pump speed: 25-30 rpm | |
| | | | | continuous | |
| | | | | Drying flux: 200 m³/h/cm² | |
| | | | | Drying temperature: 66° C. | |
| | | | | Coating: | |
| | | | | Flux: 250 m³/h/cm² | |
| | | | | Atomisation pressure: 2 BAR | |
| | | | | (200 kPa) | |
| | | | | Inlet air temperature: 66° C. | |
| | | | | Pump speed: 10-20 rpm | |
| | | | | continuous | |
| | | | | Drying flux: 350 m³/h/cm² | |
| | | | | Drying temperature: 66° C. | |
| GaleniQ ® | Povidone | Opadry ® II | Batch | Procedure 3. | Colour |
| 800 (51.0%) + | K-30 | RED 31K | 12 | Granulation 1: | homogeneity: |
| Emcompress ® | | (6.25%); | | Granulator speed: 200 rpm | Homogeneous |
| (22.3%) | | Saccharin | | Chopper speed: 250 rpm | Assay: 107.0% |
| | | (0.3%); | | Granulation liquid: 500 ml | Dissolution |
| | | Strawberry | | Granulation liquid: povidone + | 30 minutes, |
| | | flavour | | Saccharin + coating agent + | 75 rpm: 75.3% |
| | | (0.15%) | | water | 30 minutes, |
| | | | | Drying flux: 250 m³/h/cm² | 100 rpm: 91.4% |
| | | | | Drying temperature: 77° C. | |
| | | | | Granulation 2: | |
| | | | | Flux: 550 m³/h/cm² | |
| | | | | Initial pump speed: 7 rpm | |
| | | | | Granulation liquid: povidone + | |
| | | | | coating agent + water | |
| | | | | Atomisation pressure: 2 BAR | |
| | | | | (200 kPa) | |
| | | | | Inlet air temperature: 77° C. | |
| | | | | Pump speed: 35 rpm continuous | |
| | | | | Drying flux: 200 m³/h/cm² | |
| | | | | Drying temperature: 77° C. | |
| | | | | Coating: | |
| | | | | Flux: 250 m³/h/cm² | |
| | | | | Atomisation pressure: 2 BAR | |
| | | | | (200 kPa) | |
| | | | | Inlet air temperature: 52° C. | |
| | | | | Pump speed: 4 rpm continuous | |
| | | | | Drying flux: 250 m³/h/cm² | |
| | | | | Drying temperature: 52° C. | |
| GaleniQ ® | Povidone | Opadry ® II | Batch | Procedure 3. | Colour |
| 801 (51.0%) + | K-30 | RED 31K | 15 | Granulation 1: | homogeneity: |
| Emcompress ® | | (6.25%); | | Granulator speed: 200 rpm | Not Homogeneous |
| (22.3%) | | Saccharin | | Chopper speed: 250 rpm | Assay: 100.9% |
| | | (0.3%); | | Granulation liquid: 500 ml | Dissolution: N/A |
| | | Strawberry | | Granulation liquid: povidone + | |
| | | flavour | | Saccharin + coating agent + | |
| | | (0.15%) | | water | |
| | | | | Drying flux: 250 m³/h/cm² | |
| | | | | Drying temperature: 77° C. | |
| | | | | Granulation 2: | |
| | | | | Flux: 550 m³/h/cm² | |
| | | | | Initial pump speed: 7 rpm | |
| | | | | Granulation liquid: povidone + | |
| | | | | coating agent + water | |
| | | | | Atomisation pressure: 2 BAR | |
| | | | | (200 kPa) | |
| | | | | Inlet air temperature: 77° C. | |
| | | | | Pump speed: 35 rpm continuous | |
| | | | | Drying flux: 200 m³/h/cm² | |
| | | | | Drying temperature: 77° C. | |
| | | | | Coating: | |
| | | | | Flux: 250 m³/h/cm² | |
| | | | | Atomisation pressure: 2 BAR | |
| | | | | (200 kPa) | |
| | | | | Inlet air temperature: 52° C. | |
| | | | | Pump speed: 4 rpm continuous | |
| | | | | Drying flux: 250 m³/h/cm² | |
| | | | | Drying temperature: 52° C. | |
| GaleniQ ® | Povidone | Opadry ® II | Batch | Procedure 3. | Colour |
| 801 (51.0%) + | K-30 | RED 03B | 16 | Granulation 1: | homogeneity: Not |
| Emcompress ® | | (6.25%); | | Granulator speed: 200 rpm | Coloured/Not |
| (22.3%) | | Saccharin | | Chopper speed: 250 rpm | Homogeneous |
| | | (0.3%); | | Granulation liquid: 500 ml | Assay: N/A |
| | | Strawberry | | Granulation liquid: povidone + | Dissolution: N/A |
| | | flavour | | Saccharin + coating agent + | |
| | | (0.15%) | | water | |
| | | | | Drying flux: 250 m³/h/cm² | |
| | | | | Drying temperature: 77° C. | |
| | | | | Granulation 2: | |
| | | | | Flux: 550 m³/h/cm² | |
| | | | | Initial pump speed: 7 rpm | |
| | | | | Granulation liquid: povidone + | |
| | | | | coating agent + water | |
| | | | | Atomisation pressure: 2 BAR | |
| | | | | (200 kPa) | |
| | | | | Inlet air temperature: 77° C. | |
| | | | | Pump speed: 35 rpm continuous | |
| | | | | Drying flux: 200 m³/h/cm² | |
| | | | | Drying temperature: 77° C. | |
| | | | | Coating: | |
| | | | | Flux: 250 m³/h/cm² | |
| | | | | Atomisation pressure: 2 BAR | |
| | | | | (200 kPa) | |
| | | | | Inlet air temperature: 52° C. | |
| | | | | Pump speed: 4 rpm continuous | |
| | | | | Drying flux: 250 m³/h/cm² | |
| | | | | Drying temperature: 52° C. | |
| GaleniQ ® | Povidone | AquaPolish ® | Batch | Procedure 3. | Colour |
| 801 (51.0%) + | K-30 | D RED | 17 | Granulation 1: | homogeneity: Not |
| Emcompress ® | | (6.25%); | | Granulator speed: 200 rpm | Coloured/Not |
| (22.3%) | | Saccharin | | Chopper speed: 250 rpm | Homogeneous |
| | | (0.3%); | | Granulation liquid: 500 ml | Assay: NP |
| | | Strawberry | | Granulation liquid: povidone + | Dissolution: NP |
| | | flavour | | Saccharin + coating agent + | |
| | | (0.15%) | | water | |
| | | | | Drying flux: 250 m³/h/cm² | |
| | | | | Drying temperature: 77° C. | |
| | | | | Granulation 2: | |
| | | | | Flux: 550 m³/h/cm² | |
| | | | | Initial pump speed: 7 rpm | |
| | | | | Granulation liquid: povidone + | |
| | | | | coating agent + water | |
| | | | | Atomisation pressure: 2 BAR | |
| | | | | (200 kPa) | |
| | | | | Inlet air temperature: 77° C. | |
| | | | | Pump speed: 35 rpm continuous | |
| | | | | Drying flux: 200 m³/h/cm² | |
| | | | | Drying temperature: 77° C. | |
| | | | | Coating: | |
| | | | | Flux: 250 m³/h/cm² | |
| | | | | Atomisation pressure: 2 BAR | |
| | | | | (200 kPa) | |
| | | | | Inlet air temperature: 52° C. | |
| | | | | Pump speed: 4 rpm continuous | |
| | | | | Drying flux: 250 m³/h/cm² | |
| | | | | Drying temperature: 52° C. | |

N/A—not assessed
NP—not performed

By looking at the table 4 results it can be said that:
By changing the coating agent adding procedure, the granule colour homogeneity results are different: dividing the coating agent between the two granulation steps and final coating step gave rise to better granule colour homogeneity than adding it all at once in the final coating step;
By changing the inlet air flux and liquid pump speed from continuous to increasing steps, the granule assay was improved (batches Batch 3 vs. Batch 4);
By adding granulation liquid prior to granulation step (initial pump speed), the assay was increased (batches Batch 3 vs. Batch 5; batches Batch 4 vs. Batch 6);
By using a high shear granulator in granulation 1 the assay was substantially increased (batches Batch 6 vs. Batch 7);
By increasing the quantity of granulation liquid added prior to granulation step, the assay was improved, but the granule size distribution is less homogeneous;
By increasing the lactose quantity in the formulation the dissolution was improved (Batch 7 vs Batch 10 and Batch 11);
Using galenIQ® 800 instead of lactose gave rise to slower dissolution, but higher particle size granules;
By increasing the dissolution assay speed the dissolution of the formulation containing galenIQ® 800 was improved;
Opadry® 31K series gave rise to better colorization and colour homogeneity than
Opadry® 03B series (Batch 15 vs Batch 16);
Calibration of the dried granules after granulation 1 gave rise to a more homogeneous granule distribution (visual) (Batch 14);
By lowering the inlet air temperatures during all the process the colour of the granules was improved in terms of intensity and homogeneity (Batch 14).

### Conclusion

From the tested formulations the formulation corresponding to Batch 14 gave the best results. It presents adequate and homogeneous granule size, and also good assay and dissolution profiles. During granulation 2 the product temperature should be between 34 and 36° C. and during coating the product temperature should be around 38° C., this avoided sticking of the granules to the walls of the Fluid Bed Dryer and improved the granule homogeneity.

In order to obtain good colour homogeneity the addition of the coating agent is preferably divided between the granulation and coating.

### Bibliography

Handbook of pharmaceutical excipients, 4^{th} edition, American Pharmaceutical Association, 2003
European Pharmacopoeia, 6th Edition, 2008, Strasbourg
The United States Pharmacopoeia 31, The National Formulary 26, 2008, Rockville

### Summary

Pre-formulation work was performed in order to assess the most suitable excipients. After that the selected excipients were used to formulate an eslicarbazepine acetate granulate at a pilot scale.

The fillers used to perform the pre-formulation work were: Avicel® PH102, Emcompress®, Lactose 200M, GaleniQ® 800, Lactose 80M. They were combined with binders (Sodium Alginate, Starch 1500, Xanthan gum, HPMC, PEG 6000, Povidone K-30, Eudragit® RL PO). Based on the particle size (the higher the better) the chosen batches were the ones containing as a main excipient Lactose (200M and 80M), Emcompress® and GalenIQ®, and as binders xanthan gum, HPMC, sodium alginate and povidone K-30. These excipients were tested with eslicarbazepine acetate and all presented good particle size results.

Lactose 200M, Emcompress® and GalenIQ® 800/801 were used as main excipients. Povidone k-30 was used as a binder. Several Opadry colours/grades were tested as well as its addition parameters. The use of the High shear mixer in the first granulation was also tested. The following parameters were explored:

Granulation 1—Granulator speed; Chopper speed; Granulation liquid; Granulation liquid composition; Drying flow; Drying temperature;
Granulation 2—Flow; Initial pump speed; Granulation liquid composition; Atomisation pressure; Inlet air temperature; Pump speed and frequency; Drying flow; Drying temperature;
Coating—Flow; Atomisation pressure; Inlet air temperature; Pump speed; Drying flow; Drying temperature.

The following conclusions were made:
By increasing the coating agent quantity the colour homogeneity did not improve;
By changing the coating agent adding procedure, the granule colour homogeneity results were different: dividing the coating agent between the two granulation steps and final coating step gave rise to better granule colour homogeneity than adding it all at once in the final coating step;
By changing the inlet air flow and liquid pump speed from continuous to increasing steps, the granule assay was improved;
By adding granulation liquid prior to granulation step (initial pump speed), the assay was increased;
By using a high shear granulator in granulation 1 the assay was substantially increased;
By increasing the quantity of granulation liquid added prior to granulation step, the assay was improved, but the granule size distribution was less homogeneous;
Adding a disintegrant to the formulation did not improve the dissolution significantly;
By increasing the lactose quantity in the formulation the dissolution was improved;
Using galenIQ® 800/801 instead of lactose gave rise to slower dissolutions, but higher particle size granules;
By increasing the dissolution assay paddle rotation speed the dissolution of the formulation containing galenIQ® 800/801 was improved.

### Example 2

### Comparison of Granule Size of Invention with Granule Size of Tablet Granules

Batch 18 and 19 are granule compositions produced according to the invention. Batch 20 is representative of the granule size distribution of a composition that may be used in the production of tablets.

TABLE 5

| | | |
|---|---|---|
| Granules | | % Accumulated |
| Size (mm) | % Granulate | Granulate |

Batch 18

| | | | | |
|---|---|---|---|---|
| 0 | 0.0 | 0.0 | d10 | 250 |
| 63 | 0.0 | 0.0 | d50 | 420 |
| 90 | 2.5 | 2.5 | d90 | 420 |
| 180 | 6.9 | 9.4 | | |
| 250 | 30.4 | 39.8 | | |
| 420 | 52.4 | 92.3 | | |
| 520 | 7.4 | 99.7 | | |
| 710 | 0.3 | 100.0 | | |
| TOTAL | 100.0 | | | |

Batch 19

| | | | | |
|---|---|---|---|---|
| 0 | 0.0 | 0.0 | d10 | 250 |
| 63 | 0.0 | 0.0 | d50 | 420 |
| 90 | 2.4 | 2.5 | d90 | 420 |
| 180 | 3.1 | 5.6 | | |
| 250 | 35.3 | 40.9 | | |
| 420 | 53.30 | 94.2 | | |
| 520 | 5.8 | 100.0 | | |
| 710 | 0.0 | 100.0 | | |
| TOTAL | 100.0 | | | |

Batch 20 - Tablet Granules

| | | | | |
|---|---|---|---|---|
| 0 | 1.0 | 1.0 | d10 | 90 |
| 63 | 2.0 | 3.0 | d50 | 250 |
| 90 | 11.0 | 14.0 | d90 | 520 |
| 180 | 16.0 | 30.0 | | |
| 250 | 21.0 | 51.0 | | |
| 420 | 32.0 | 83.0 | | |
| 520 | 15.0 | 98.0 | | |
| 710 | 3.0 | 101.0 | | |
| TOTAL | 101.0 | | | |

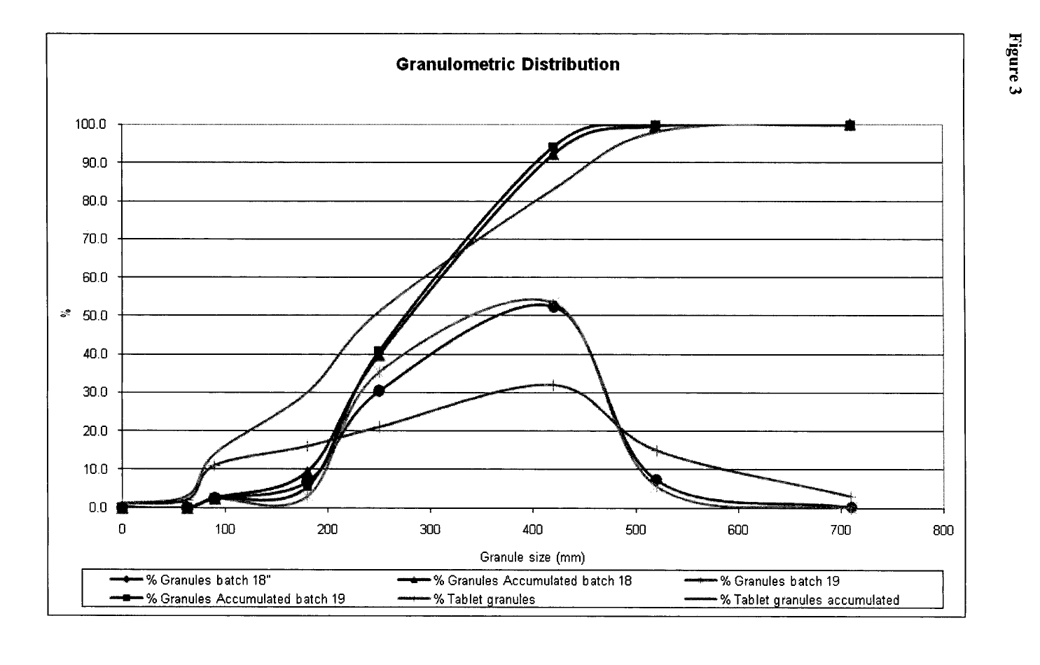

A graphical representation of the granule size distribution of these batches is shown in FIG. 3.

### Example 3

### Additional Data Relating to Granules of the Invention Compared to Tablet Granules

A laboratorial scale batch of 700 sachets was manufactured using the same formulation of the oral granules as described above (Batch 19).

The manufacturing process for the tablet granulation process is:
1—Mix povidone with purified water until complete dissolution is achieved, then add the saccharin and a portion of the Opadry and mix until a homogeneous suspension is achieved (granulation liquid);
2—Mix the other components in the laboratorial mixer granulator;
3—Add the granulation liquid and granulate in the laboratorial mixer granulator; and
4—Dry the granules in a fluid bed dryer.

This batch (Batch 21) was then compared to a batch manufactured using the process of the invention (Batch 19). The following results were obtained:

| | | Batch 19 | Batch 21 |
|---|---|---|---|
| Appearance | | Homogeneous | Non homogeneous |
| | | coloured | coloured granules |
| | | red granules | and powder |
| D₀ | | 0.64 | 0.67 |
| D₁₂₅₀ | | 0.73 | 0.75 |
| Hausner ratio | | 1.07 | 1.07 |
| Carr Index | | 9.32 | 9.60 |
| Flow speed (g/s) | | 7.4 | 6.5 |
| Angle of repose (°) | | 28.5 | 31.8 |
| Particle | d(0.1) | 207.8 | 133.4 |
| size | d(0.5) | 361.7 | 636.8 |
| distribution | d(0.9) | 610.6 | 1292.5 |
| (μm) | d(0.95) | 688.3 | 1493.8 |

| | | |
|---|---|---|
| Dissolution (%) | 91.1 | 89.5 |

Analyzing the results, the appearance of the two batches was very different. Batch 19 was perceived as homogeneous coloured red granules, while Batch 21 was perceived as granules and powder which, depending on the particle, were coloured from white to red.

For the values of the density, Hausner ratio and Carr Index, no significant differences were found between the batches. The flow speed and angle of repose are better for Batch 19 which shows that its flowability is improved relative to the tablet granules.

The particle size distribution is very different between the two batches. Batch 19 has a narrower distribution (480.5 μm between the extremes) compared to Batch 21 (1360.4 μm between the extremes). As noted above Batch 19 was perceived as granules and Batch 21 was perceived as a mixture of powder with granules of different sizes.

In summary, some of the advantages of the process batch of the invention (Batch 19) over a typical tablet granulation process batch (Batch 21) are:
Production of homogeneous colored granules;
Narrower particle size distribution;
Better flowability.

### Experimental Protocols

The protocols for measuring the dissolution, flow and apparent volume of the granules are described below.

### Protocol for Measuring Apparent Volume

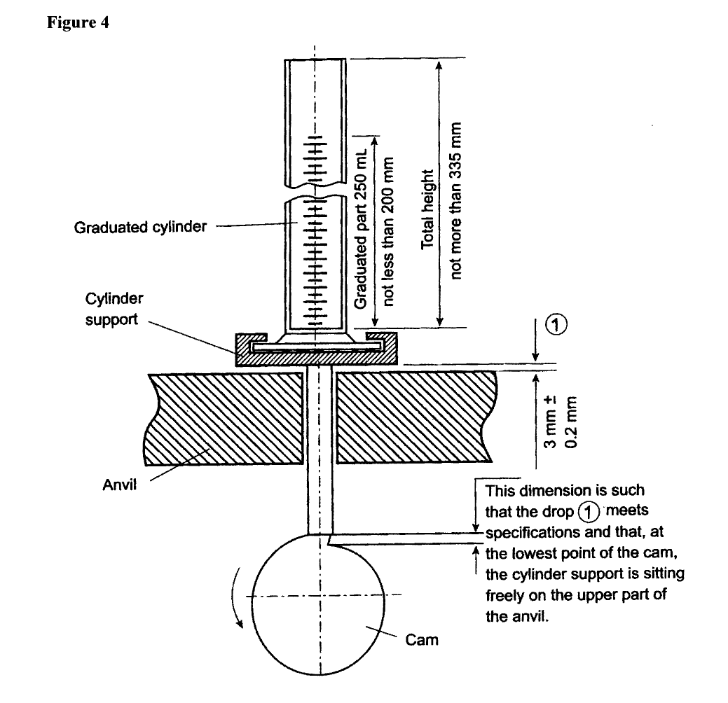

### Equipment (See FIG. 4)

Erweka SVW
250 ml beaker with 2 ml graduation

### Procedure

Perform the following procedure in triplicate using the equipment shown in FIG. 4:
1. Turn on the Erweka SVW equipment.
2. Weigh a sample of about 100 g, record the value (Determination of the Apparent Volume) and place the sample in the beaker with the aid of a funnel. If it is impossible to place 100.0 g of sample in the beaker, choose a sample whose apparent volume is between 50 and 250 ml and write down the mass.
3. Measure the initial volume (V₀—bulk volume, in ml) and record the value.
4. Program beats register on the Erweka SVW to 10 and press START.
5. Measure the volume after 10 beats (V₁₀, in ml) and record the value.
6. Press RESET, Program beats register on the Erweka SVW to 490 and press START.
7. Measure the volume after 500 beats (V₅₀₀, in ml) and record the value.
8. Press RESET, Program beats register on the Erweka SVW to 750 and press START.
9. Measure the volume after 1250 beats (V₁₂₅—tapped volume in ml) and record the value.
10. If the difference between V₅₀₀ and V₁₂₅₀ is more than 2 ml, perform an extra 1250 beats and record the value (V₂₅₀₀—v tapped volume in ml).

### Calculation

From the previously obtained results perform the following calculations:

| Density | Compactation | Compressibility | | |
|---|---|---|---|---|
| (g/ml) | capacity (ml) | Index (%) | Hausner Ratio | Carr Index |
| $\begin{matrix}{D}_{0} = \frac{m}{{V}_{0}} \\ {D}_{10} = \frac{m}{{V}_{10}} \\ {D}_{500} = \frac{m}{{V}_{500}} \\ {D}_{1250} = \frac{m}{{V}_{1250}} \\ {D}_{25000} = \frac{m}{{V}_{2500}}\end{matrix}$ | V₁₀ − V₅₀₀ | $IC = \frac{{V}_{10} - {V}_{500}}{{V}_{10}} * 100$ | $HausnerRatio = \frac{{D}_{500}}{{D}_{10}} * 100$ | $CarrIndex = \frac{{D}_{500} - {D}_{0}}{{D}_{500}} * 100$ |

D₀—Apparent density
D₁₀—Density after 10 beats
D₅₀₀—Density after 500 beats
D₁₂₅₀—Tapped density
D₂₅₀₀—Tapped density

### Protocol for Measuring Flow

### Equipment

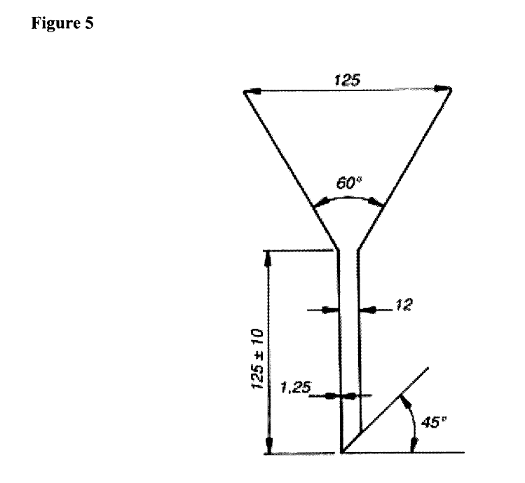

Pharma Test™—PTG
Funnel with the dimensions shown in FIG. 5 in cm.
Funnel support
Milimetric paper sheet
Chronometer
Caliper rule

### Determination by the Pharma Test Method

1. Turn on the Pharma Test™—PTG equipment and select program 1.
2. Place the funnel in the device.
3. Weigh a 100.0 g sample, record the weight, and place it in the funnel. If a 100 g sample occupies more than the funnel capacity, weigh a smaller sample and record the weight.
4. Press START.
5. Record the values for the flow time (t) in (s) and angle of repose (α) in degrees (°).
6. Place the powder in the funnel again and repeat the procedure two more times. Calculate the average values for weight, flow time and angle of repose, and record them.
   Determination by the Funnel Method Described in the Ph. Eur.

Perform the procedure described below in triplicate:
1. Place the funnel in its holder at a height of 7 cm.
2. Place under the funnel a milimetric paper sheet or other device which allows a correct reading of the base of the powder cone to be formed (example: a petri plaque on top of a laboratory cup).
3. Place a 100 g sample in the funnel and block the powder exit. Record the weight of the sample. If a 100 g sample occupies more than the funnel capacity, weigh a smaller sample and record the weight.
4. Unblock the powder exit and let the powder fall on the milimetric paper. Record the time which the powder takes to completely empty the funnel (t) in seconds (s).
5. A powder cone is formed, delimit the cone base with a pen and measure its diameter (d) in cm. Record the value.
6. Measure the height of the cone, in cm, using the caliper rule and record the value.
7. From the previous results calculate the flow speed and angle of repose using the following formulas:

$$Flow {Speed}_{( g / s )} = \frac{m}{t}$$

$$Angle of Rpose = Arctg ( \frac{h}{r} )$$

h—height of the cone;
r—radius of the cone base (d/2).
8. Repeat the previous steps two more times, calculate the average values and record them.
   Protocol for Measuring Dissolution of Eslicarbazepine Acetate from a Composition

### Procedure

Rotating paddle apparatus (apparatus 2; section 2.9.3 of the Ph. Eur. and chapter <711> of the USP)
Dissolution medium HCl 0.01 mol/l
Volume 1000 ml (±1%)
Temperature 37.0±0.5° C.
Stirrer speed 100±4 rpm
Duration of test 30 minutes

### Instrumental Technique

Reversed Phase-HPLC-UV detection
Eluent A MilliQPlus ultra-pure water filtered through a 0.45 μm membrane
Eluent B Acetonitrile HPLC grade
Column Merck chromolith RP-18e, 100-4.6 mm or equivalent

Flow rate 1.0 ml/min

Detection 250 nm

Injection volume 20 μl

Column temperature 30° C.

Mobile phase Isocratic; eluent A/eluent B (70:30) (v/v)

### Example 4

### Stability of Granule Compositions

Granule compositions in accordance with the invention were tested for stability.

The stability of Batches 18 and 19 described above were tested both in and outside of sachets. The sachets were tested at 25° C./60% RH at 0, 3, 6, 9 and 12 months. The sachets were also tested at 40° C./75% RH at 0, 3 and 6 months. The granules presented satisfactory stability in terms of photostability, impurity levels and assay of API.

### Example 5

### Colour Homogeneity of Granule Compositions

When the granules contain a colouring agent, this colouring agent can be distributed in the composition so that the granules have a homogeneous colour across their cross section.

This homogeneous colour allows assessment of whether the process used to produce the granules has been carried out correctly. Therefore, any problems in the production process can be identified relatively easily and quickly.

The granules may also have a homogeneous colour as a whole so that each granule is substantially the same colour as the other granules.

This homogeneous colour again allows quick and easy assessment of the production process. If not all the granules have a homogeneous colour, this can indicate a problem with the production process.

It has also been shown that granules having a homogeneous colour from one granule to another are more appealing to a subject and are an empirical measure of product quality. This means that the granules are likely to be more acceptable to the subject and may help with patient compliance.

1. A solid pharmaceutical composition, the composition comprising eslicarbazepine acetate and one or more pharmaceutically acceptable excipients, wherein the composition is in the form of granules, and wherein at least 90% of the granules of the composition have a particle size of 90 μm or more, and/or wherein at least 50% of the granules of the composition have a particle size of 250 μm or more.

2-5. (canceled)

6. The composition of claim 1, wherein at least 90% of the granules of the composition have a particle size of 1600 μm or less.

7-9. (canceled)

10. The composition of claim 1, wherein at least 80% of the granules of the composition have a particle size which falls within a range selected from 1500 μm, 1000 μm, 600 μm, 300 μm, and 200 μm.

11-14. (canceled)

15. The composition of claim 1, comprising between about 5% and about 85% by weight of eslicarbazepine acetate.

16-18. (canceled)

19. The composition of claim 1, comprising between about 15% and about 95% filler material by weight.

20-30. (canceled)

31. The composition of claim 1, comprising between about 2% and about 15% binder by weight.

32-33. (canceled)

34. The composition of claim 1, wherein the composition further comprises a colouring agent, and wherein the granules have a homogeneous colour across their cross section.

35. The composition of claim 34, comprising between about 1% and about 20% colouring agent by weight.

36. The composition of claim 1, wherein the composition further comprises a flavouring agent.

37. The composition of claim 36, comprising between about 0.05% and about 5% flavouring agent by weight.

38. The composition of claim 1, further comprising a sweetener.

39. The composition of claim 38, further comprising between about 0.1% and about 10% sweetener by weight.

40. (canceled)

41. The composition of claim 1, comprising between about 5% and 40% by weight of eslicarbazepine acetate, between about 40% and about 80% filler material by weight, between about 2% and about 15% povidone by weight, and between about 1% and about 10% colouring agent by weight, wherein the filler material comprises lactose and/or dibasic dihydrate calcium phosphate and/or mannitol, and wherein the granules have a homogeneous colour across their cross section.

42. A process for producing a granular composition comprising a pharmaceutically active agent, the process comprising:
(1) granulating a mixture comprising a pharmaceutically active agent and one or more pharmaceutically acceptable excipients using a first granulation liquid;
(2) drying the granules formed in (1);
(3) optionally, calibrating the size of the granules resulting from (2);
(4) granulating the granules resulting from (2) or (3) using a second granulation liquid;
(5) drying the granules formed in (4);
(6) coating the granules resulting from (5) using a coating liquid; and
(7) drying the coated granules formed in (6),
wherein at least 90% of the coated granules that are produced have a particle size of 90 μm or more and/or wherein at least 50% of the coated granules that are produced have a particle size of 250 μm or more.

43. The process of claim 42, wherein the pharmaceutically active agent is eslicarbazepine acetate.

44. The process of claim 42, wherein the granulation in (1) takes place in a high shear granulator.

45. The process of claim 42, wherein the granulation in (1) and the drying of the granules in (2) take place in a fluid bed dryer.

46. The process of claim 42, wherein the drying of the granules in (1) takes place in a fluid bed dryer.

47. The process of claim 42, wherein (4) is carried out in a fluid bed dryer.

48. The process of claim 42, wherein (6) is carried out in a fluid bed dryer.

49. The process of claim 42, wherein the first granulation liquid, the second granulation liquid and the coating liquid comprise a colouring agent.

50. The process of claim 42, wherein drying of the granules in one or more of the steps involves drying the granules until the granule relative humidity is below about 3%.

51. The process of claim 42, wherein the first and second granulation liquids are aqueous solutions comprising a binder selected from xanthan gum, HPMC, starch, sodium alginate and povidone.

52. The process of claim 42, wherein the first and second granulation liquids are aqueous solutions comprising povidone.

53. The process of claim 42, wherein, when the drying in each step is carried out in a fluid bed dryer, the drying of the granules in each step takes place at an inlet air and granule temperature between about 50° C. and about 80° C.

54. The process of claim 45, wherein drying of the granules in each step takes place at a drying flux of between about 20% and about 90% of the fluid bed dryer maximum flux capacity.

55. (canceled)

56. The process of claim 42, wherein the first and second granulation liquids are added prior to commencement of the respective granulation steps.

57. The process of claim 42, wherein the rate of introduction of the first and second granulation liquids is increased over time.

58. The process of claim 45, wherein, when a fluid bed dryer is used to carry out (1), (4) and (6), the rate of introduction of the first and second granulation liquids, and the coating liquid is between about 0.02% and about 1% of the fluid bed dryer total volume/minute.

59. The process of claim 45, wherein, when a fluid bed dryer is used to carry out (1), (4) and (6), air is used to transport the first and second granulation liquids, and the coating liquid into the fluid bed dryer.

60. The process of claim 59, wherein the pressure of the transport air is between about 0.1 bar (10 kPa) and about 6 bar (600 kPa).

61. The process of claim 42, wherein air flow during granulation or coating is increased in a stepwise manner over time.

62. The process of claim 42, wherein when a fluid bed dryer is used to carry out (4), the air flow during granulation is between about 10% and about 100% of the fluid bed dryer maximum flux capacity.

63. The process of claim 42, wherein, when a fluid bed dryer is used to carry out (1), (4) and (6), the temperature of the inlet air entering the fluid bed dryer during granulation or coating is between about 30° C. and about 80° C.

64. The process of claim 42, wherein the temperature of the mixture during granulation in (1) and/or of the granules in (4) or (6) is between about 10° C. and about 70° C.

65. The process of claim 42, wherein (3) comprises screening the granules resulting from (2) to ensure the particles have a particle size of about 2 mm or less.

66. (canceled)

67. A granular composition produced by the process of claim 42.

68. (canceled)

69. A method of treating or preventing a disorder selected from epilepsy, neuropathic pain, migraine, fibromyalgia and affective disorders, comprising administering to a subject in need thereof an effective amount of a solid pharmaceutical composition, the composition comprising eslicarbazepine acetate and one or more pharmaceutically acceptable excipients, wherein the composition is in the form of granules, and wherein at least 90% of the granules of the composition have a particle size of 90 μm or more, and/or wherein at least 50% of the granules of the composition have a particle size of 250 μm or more.

70-71. (canceled)
